(19) United States
(12) Patent Application Publication
(10) Pub. No.: US 2020/0308568 A1
(43) Pub. Date: Oct. 1, 2020

(54) ALCOHOL STABLE ENZYMES

(71) Applicant: BioHybrid Solutions LLC, Pittsburgh, PA (US)

(72) Inventors: Antonina Simakova, Pittsburgh, PA (US); Gregory Lewis, Pittsburgh, PA (US); Alan Russell, Wexford, PA (US); Krzysztof Matyjaszewski, Pittsburgh, PA (US)

(21) Appl. No.: 16/828,625

(22) Filed: Mar. 24, 2020

Related U.S. Application Data
(60) Provisional application No. 62/824,751, filed on Mar. 27, 2019.

(51) Int. Cl. C12N 9/96; C12N 9/20; A61K 47/58; A61K 9/08; A61K 47/10; C08F 293/00; A61K 38/46

(52) U.S. Cl. CPC C12N 9/96; C12N 9/20; C12Y 301/01003; A61K 47/58; C08F 2438/01; A61K 47/10; C08F 293/005; A61K 38/465; A61K 9/08

(57) ABSTRACT

The present disclosure provides enzyme-polymer conjugates stable to denaturing action of alcohols and methods for using enzyme-polymer conjugates in biocatalysis.

### CROSS-REFERENCES TO RELATED APPLICATIONS

This application claims the benefit of U.S. Provisional Patent Application No. 62/824,751, filed Mar. 27, 2019, which is entirely incorporated herein by reference for all purposes.

### STATEMENT REGARDING FEDERALLY SPONSORED RESEARCH OR DEVELOPMENT

This invention was made with government support under Award No. 1746912 awarded by the National Science Foundation (NSF). The government has certain rights in the invention.

### BACKGROUND

Enzymes are nature's catalysts—they accelerate complex chemical reactions within cells and are required to sustain life. Harnessing the power of enzyme catalysts for complex syntheses (biocatalysis) has implications in diverse fields, ranging from therapeutics to advanced materials to commodity biofuels. Enzymes are biodegradable and operate under mild, safe reaction conditions, paving the way toward more sustainable chemical processing. Higher purity products from biocatalysis reduce the costly need for product purification. Despite the advantages of enzymatic biocatalysis, industrial adoption has been hampered by low enzyme stability under typical chemical processing conditions. Many function best at near-neutral pH and will denature upon exposure to solvents, heat, acidic or basic conditions. Short-chain aliphatic alcohols are commonly utilized co-solvents and reagents in biocatalysis. But such alcohols hamper the activity of enzymes when used at concentrations beneficial for biocatalytic reactions due to irreversible denaturation of enzymes.

### SUMMARY

As such, there is a need for alcohol tolerant enzymes and biomolecules with enhanced properties for use in industrial biocatalysis. In certain aspects, the present disclosure provides an enzyme-polymer conjugate comprising: at least one polymer covalently bound to an enzyme, optionally via a covalent linker; wherein the at least one polymer is selected from pHEAAm, pDMAAm, pTRISAAm, pNIPAAm, pCBAAm, pSBAAm, pAMPSA, pAMP, pAAm and pDEAAm, or a combination thereof and wherein the enzyme is a lipase.

The at least one polymer may be selected from pHEAAm, pDMAAm, pTRISAAm and pNIPAAm, or a combination thereof. For example, the at least one polymer may be selected from pHEAAm and pNIPAAm. The at least one polymer may be covalently bound to a lysine residue of the enzyme, optionally via a covalent linker. In some embodiments, the covalent linker comprises

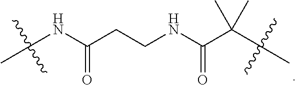 [SMILES: *NC(=O)CCNC(=O)C(*)(C)C]

Optionally, at least 30% of the total lysine residues of the enzyme are conjugated to a polymer. The conjugate may comprise a plurality of polymers. Optionally, the plurality of polymers is made by growing the polymers directly from the surface of the protein using atom-transfer radical polymerization (ATRP). Each polymer in the plurality of polymers may comprise monomeric units of the same type, or each polymer in the plurality of polymers may comprise a first polymer and a second polymer, wherein the first polymer and the second polymer each comprise monomeric units of a different type. The at least one polymer may comprise 10 to 200 monomeric units.

In some embodiments, the lipase originates from Candida Antarctica A (CALA). The conjugate may exhibit at least 1 μmol/min/mg of enzymatic activity after 1 hour of incubation in an aqueous solution comprising at least 50% by volume of a C₁₋₃ alcohol. The conjugate may exhibit at least 1 μmol/min/mg of enzymatic activity after 24 hours of incubation in an aqueous solution comprising at least 50% by volume of a C₁₋₃ alcohol.

In certain aspects, the present disclosure provides a method of catalyzing a reaction, comprising: combining an enzyme-polymer conjugate with a substrate for the enzyme in an aqueous solution comprising at least 20% by volume of a C₁₋₆ alcohol, thereby forming a reaction mixture; wherein the enzyme-polymer conjugate comprises at least one polymer covalently bound to the enzyme, optionally via a covalent linker. The method may further comprise separating a product from the reaction mixture. Optionally, the C₁₋₆ alcohol is a C₁₋₃ alcohol, such as methanol, ethanol, 1-propanol, 2-propanol, or a combination thereof. Optionally, the enzyme is a lipase. The at least one polymer may be a zwitterionic polymer, a hydrophilic polymer, or a temperature-responsive polymer. Optionally, the at least one polymer is selected from pHEAAm, pDMAAm, pTRISAAm, pNIPAAm, pCBAAm, pSBAAm, pAMPSA, pAMP, pAAm and pDEAAm, or a combination thereof. In some embodiments, the at least one polymer is selected from pHEAAm, pDMAAm, pTRISAAm and pNIPAAm, or a combination thereof. The at least one polymer may be selected from pHEAAm and pNIPAAm.

The at least one polymer may be covalently bound to a lysine residue of the enzyme, optionally via a covalent linker. The covalent linker may comprise

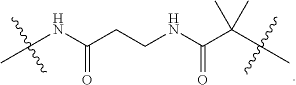 [SMILES: *NC(=O)CCNC(=O)C(*)(C)C]

In some embodiments, at least 30% of the total lysine residues of the enzyme are conjugated to a polymer. The conjugate may comprise a plurality of polymers, optionally wherein the plurality of polymers is made by growing the polymers directly from the surface of the protein using atom-transfer radical polymerization (ATRP). Each polymer in the plurality of polymers may comprise monomeric units of the same type, or each polymer in the plurality of polymers may comprise a first polymer and a second polymer, wherein the first polymer and the second polymer each comprise monomeric units of a different type. Optionally, the at least one polymer comprises 10 to 200 monomeric units. The enzyme may be an esterase, lipase, transferase, oxidoreductase or protease, such as a lipase. Optionally, the lipase originates from Candida Antarctica A (CALA). In some embodiments, the conjugate exhibits at least 1 μmol/min/mg of enzymatic activity.

In certain aspects, the present disclosure provides a solution comprising: an aliphatic alcohol, wherein the alcohol comprises no greater than 75 percent water by volume as a co-solvent; and an enzyme-polymer conjugate that retains at least 30 percent of the activity of the native enzyme in water; wherein the enzyme-polymer conjugate comprises covalently bound polymer chains grown from an enzyme-initiator conjugate. The solution may comprise greater than 25 percent methanol, ethanol, 1-propanol, or 2-propanol, or combination of any thereof. Optionally, the enzyme-polymer conjugate was prepared by conjugating a protein-reactive polymerization initiator to the enzyme and subsequently polymerizing a plurality of monomers to form a polymer chain on the enzyme. The enzyme-polymer conjugate may comprise a lipase-polymer conjugate, a transaminase-polymer conjugate, a ketoreductase-polymer conjugate, or a glucose dehydrogenase-polymer conjugate. The enzyme-polymer conjugate may retain its activity for at least one hour, such as between 1 and 24 hours. In some embodiments, the enzyme-polymer conjugate comprises an enzyme capable of catalyzing a hydrolysis reaction, a transesterification reaction, a redox reaction, a group-transfer reaction, a condensation reaction, a polyester synthesis reaction, or a combination of any thereof.

Optionally, the enzyme-polymer conjugate comprises poly(N-hydroxyethyl acrylamide) covalently bound to the enzyme. The enzyme-polymer conjugate may comprise covalently bound to the enzyme poly(N-hydroxyethyl acrylamide), poly(N-isopropylacrylamide), poly(4-Acryloylmorpholine), poly(carboxybetaine acrylamide), poly(dimethyl acrylamide), poly(diethyl acrylamide), or a combination thereof. The enzyme-polymer conjugate may comprise covalently bound to the enzyme poly(2-hydroxyethyl methacrylate), poly(2-hydroxyethyl acrylate), or a combination thereof. The polymer of the enzyme-polymer conjugate may impart stability and activity of the enzyme in the presence of an alcohol.

In certain aspects, the present disclosure provides a reaction mixture comprising: at least 20% by volume of a C₁₋₆ alcohol in water; an enzyme-polymer conjugate described herein; and a substrate of the enzyme.

In certain aspects, the present disclosure provides a method of stabilizing enzymes against denaturation and retaining activity in the presence of alcohols. The method may comprise (a) modifying an enzyme with controlled radical initiator group; (b) growing synthetic polymer from the enzyme-initiator conjugate; (c) utilizing enzyme-polymer conjugate in alcohol mixtures with water, where water content is not higher than 75% by volume, and (d) where enzyme-polymer conjugate retains at least 30% of activity of fully active unmodified enzyme.

In some embodiments, a method disclosed herein may further comprise use of the enzyme-polymer conjugate in the presence of methanol, ethanol, n-propanol, isopropanol, or any combination thereof, where alcohol content is high enough to cause irreversible enzyme denaturation. The enzyme-polymer conjugates may be used in homogenous or heterogeneous solutions, slurries, emulsions, and/or mixtures containing combinations of water and alcohols. Use of enzyme-polymer conjugates in a variety of solvents and solvent mixtures allows for use in industrial biocatalysis, which relies on enzymatic biocatalysis performed in combinations of solvents to maximize reaction yields and conversion. Methanol, ethanol, n-propanol, and isopropanol are among the most common co-solvents used for biocatalysis, which result in enzyme denaturation. In some embodiments, each different biocatalysis reaction may require a unique combination of enzyme-polymer conjugate properties, including but not limited to, polymer type(s), number of polymers per enzyme, structure of the polymer(s), and size of each polymer.

In embodiments, the polymer that provides stabilization to an enzyme in methanol and ethanol comprises a monomer having a hydroxyl group in its side chain. Additionally, the polymer that provides stabilization to an enzyme in n-propanol and isopropanol may comprise a monomer having a propyl or isopropyl group in its side chain. The monomer may be selected from a (meth)acrylate and a (meth)acrylamide. In some embodiments, the polymer comprises a mixture of at least two monomers selected from a (meth)acrylate and a (meth)acrylamide. The growing of (b) may comprise providing a first and a second monomer to the enzyme-initiator conjugates. The second monomer may be selected from a (meth)acrylate and a (meth)acrylamide. A (meth)acrylate and a (meth)acrylamide described herein may comprise one or more of a carboxybetaine, a sulfonate, a quaternary ammonium, a dialkylamino, an amino, a carboxylate, a hydroxyl, a sulfoxy or an oligo(ethylene glycol) moiety. In some embodiments, the monomer comprises a meth(acrylate) or a (meth)acrylamide, wherein the (meth)acrylate or the (meth)acrylamide comprises at least one of a sulfonate anion and an ammonium cation.

In practicing any of the subject methods, the enzyme-polymer conjugate may comprise a covalently attached polymer including but not limited to, poly(N-hydroxyethyl acrylamide), poly(N-hydroxymethyl acrylamide), poly(N-isopropylacrylamide), poly(4-Acryloylmorpholine), poly(carboxybetaine acrylamide), poly(dimethyl acrylamide), poly(diethyl acrylamide), poly(2-hydroxyethyl methacrylate), poly(2-hydroxyethyl acrylate), or any combination thereof. The polymer selected for covalent binding to the enzyme may impart stability and/or activity to the enzyme in the presence of an alcohol or a combination of alcohols. In some embodiments, the covalently bound polymer, or combination thereof, is selected for specific biocatalysis reaction conditions and solvent combinations.

In practicing any of the subject methods, the enzyme-polymer conjugate may contain a plurality of polymers covalently bound to the enzyme. The number of polymers bound to an enzyme is an integer from 1 to X, wherein X is the total number of polymerization initiator sites available on the surface of the enzyme. In certain aspects, the number of polymerization initiator sites may be conjugated with initiator in such a way that some, or even all available sites are available for polymerization, wherein the conjugation conditions determine the degree of modification.

In practicing any of the subject methods, the controlled radical polymerization initiator may comprise an activated ester, alkyl halide or chain transfer agent. Optionally, the controlled radical polymerization initiator is a compound of Formula (I):

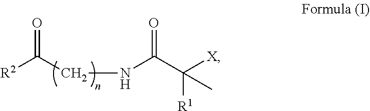

wherein X is a halogen or a chain transfer agent; R¹ is hydrogen or alkyl; R² is an active ester moiety; and n is an integer from 1 to 6. For a compound of Formula (I), X may be Cl, Br or F. Optionally, the controlled radical polymerization initiator is a compound of Formula (II):

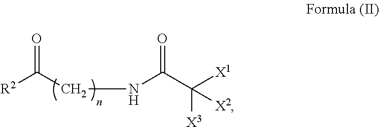

wherein X′ is halogen or a chain transfer agent; X² is alkyl, aryl, halogen or a chain transfer agent; X³ is hydrogen, halogen or alkyl; R² is an active ester moiety; and n is an integer from 1 to 6. For a compound of Formula (II), X¹ may be Cl, Br or F. In some embodiments, X² is C₁₋₆ alkyl, phenyl, halogen or a chain transfer agent, such as X² is methyl, phenyl, halogen or a chain transfer agent. In some embodiments, X² is Cl, Br or F. In some embodiments, X³ is hydrogen, halogen or C₁₋₆ alkyl.

Any enzyme-initiator conjugate described herein may comprise a peptide or a protein. Optionally, the enzyme-initiator conjugate is a compound of Formula (III):

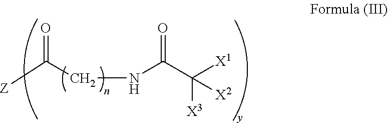

wherein Z is the enzyme; y is an integer from 1 to 100; X¹ is halogen or a chain transfer agent; X² is methyl, aryl, halogen or a chain transfer agent; X³ is hydrogen, halogen or alkyl; R² is an active ester moiety; and n is an integer from 1 to 6. In some embodiments, X² is methyl, phenyl, halogen or a chain transfer agent.

In certain embodiments, the enzyme-polymer conjugate may contain a covalently bound polymer with a linear, branched, dendritic, brush-like, cross-linked, or networked structure. The type of polymer structure may be determined by the selected monomer, through the addition of cross-linking reagents, post-polymerization modification, or other such methods. Polymer structure may impart stability and/or activity to the enzyme in the presence of an alcohol or a combination of alcohols.

In practicing any of the subject methods, the size of the covalently bound polymer may be selected to impart stability and/or activity to the enzyme in the presence of an alcohol or a combination of alcohols. The polymer may comprise a plurality of monomer, or combination thereof, with a size proportional to the number of monomers, wherein the number of monomers is an integer between 1 and 1000. The polymer size may be controlled by selection of polymerization reaction conditions or post-polymerization modification.

In embodiments, the enzyme-polymer conjugate comprises an enzyme or combination of enzymes capable of catalyzing biochemical reactions, including but not limited to, hydrolysis reactions, transesterification reactions, reduction-oxidation reactions, functional group transfer reactions, condensation reactions, polyester synthesis reactions, or any combination thereof. Selection of enzyme-polymer conjugates may be determined by reaction conditions necessary for performing the targeted biochemical reactions. The selected enzyme-polymer conjugate may comprise, but is not limited to, a lipase-polymer conjugate, a transaminase-polymer conjugate, a ketoreductase-polymer conjugate, and/or a glucose dehydrogenase-polymer conjugate.

In embodiments, the enzyme-polymer conjugate provides stabilization in an alcohol-water mixture for an amount of time equal to one hour, greater than one hour, between one and twenty-four hours, or greater than twenty-four hours. Typical industrial biocatalysis reactions require lengths of time greater than one hour, often exceeding twenty-four hours. Reactions of this nature may require exposure of enzymes to alcohol containing solvent for some or even all of the reaction duration, as such enzyme-polymer conjugates must provide stabilization properties exceeding the biochemical reaction duration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the synthesis of biomolecule-polymer conjugates involving (A) attachment of initiator to biomolecule to form a biomolecule-initiator conjugate and (B) polymerization of monomers to the biomolecule-initiator conjugate to form a biomolecule-polymer conjugate.

FIG. 2 illustrates the synthesis of enzyme-polymer conjugates utilizing “grafting to” process, where preformed polymer is attached to a biomolecule.

FIG. 3 illustrates the synthesis of enzyme-initiator (A) and shows covalent bond structure (B) between ATRP initiating moiety and an enzyme.

FIG. 4 illustrates application of ATRP method to “grafting from” an enzyme.

FIG. 5 illustrates a high-throughput polymer bioconjugation platform for application in biocatalysis.

FIG. 6 illustrates the decrease in activity of lipase CALA upon incubation in mixtures of alcohols with buffered water for 24 hr at 30° C.

FIG. 7 illustrates structures of monomers that were tested in preparation of enzyme-polymer conjugates.

FIG. 8 illustrates a prediction profiler for polydispersity of BSA-polyacrylamide

FIG. 9 illustrates the effect of monomer type and labeling density on DLS diameter and retained hydrolysis kinetic activity as compared to unmodified lipase CALA.

FIG. 10 illustrates the effect of monomer type on DLS diameter and retained hydrolysis kinetic activity after incubation in 50% methanol for 24 hours at 30° C.

FIG. 11 illustrates a structure of a lipase-pHEAAm conjugate.

FIG. 12 illustrates the effect of monomer type and labeling density on DLS diameter and retained hydrolysis kinetic activity after incubation in 75% methanol for 24 hours at 30° C. as compared to unmodified lipase CALA.

FIG. 13 provides comparisons between enzyme-polymer conjugate kinetic activity in pNPB assay after incubation in 75% alcohol solution (Stability) and end-point activity in pNPB assay containing 75% alcohol, after incubation in 75% same alcohol solution (Activity).

FIG. 14 illustrates a comparison of lipase catalyzed biotransformations with enzyme-polymer conjugates. Methyl oleate was synthesized by oleic acid esterification with methanol. Isopropyl acetate was synthesized via vinyl acetate transesterification with isopropanol.

FIG. 15 depicts a proton NMR spectra of the products of the reaction of vinyl acetate transesterification in isopropanol catalyzed by unmodified lipase CALA.

FIG. 16 depicts a proton NMR spectra of the products of the reaction of vinyl acetate transesterification in isopropanol catalyzed by lipase CALA—pNIPAAm conjugate.

### DETAILED DESCRIPTION

### Definitions

The term “biomolecule” refers to a protein or enzyme. The terms “protein” and “enzyme” are used interchangeably herein to refer to a polymer of amino acids.

The term “controlled radical polymerization initiator” refers to a molecule that generates a radical species to begin the synthesis of a polymer chain by successive addition of free-radical building blocks. The terms “controlled radical polymerization initiator” and “initiator” are used interchangeably herein to refer to a molecule that begins a radical polymerization process.

The term “enzyme-initiator conjugate” refers to a complex that comprises both an enzyme and one or more controlled radical polymerization initiators, such as five or more, 10 or more, 25 or more, 50 or more, or 100 or more controlled radical polymerization initiators. Preferably, the one or more controlled radical polymerization initiators are covalently attached to the enzyme. As used herein, an enzyme-initiator conjugate is a specific type of biomolecule-initiator conjugate, wherein the biomolecule is an enzyme.

The term “enzyme-polymer conjugate” refers to any complex that comprises both an enzyme and one or more polymer chains, such as five or more, 10 or more, 25 or more, 50 or more, or 100 or more polymer chains. Preferably, the one or more polymer chains are covalently attached to the biomolecule. As used herein, an enzyme-polymer conjugate is a specific type of biomolecule-polymer conjugate, wherein the biomolecule is an enzyme.

The term “atom transfer radical polymerization” (ATRP) refers to a polymerization technique that forms carbon-carbon bonds via a transition metal catalyst.

The terms fourier transform infrared, near-infrared, high-performance liquid chromatography, gas chromatography, nuclear magnetic resonance, mass spectroscopy and gel permeation chromatography are referred to as FTIR, NIR, HPLC, GC, NMR, MS, and GPC, respectively. Monomeric N-isopropylacrylamide is referred to as NIPAAm and poly(N-isopropylacrylamide) is referred to as pNIPAAm. Monomeric N-hydroxyethyl acrylamide is referred to as HEAAm and poly(N-hydroxyethyl acrylamide) is referred to as pHEAAm. Monomeric N-hydroxymethyl acrylamide is referred to as HMAAm and poly(N-hydroxymethyl acrylamide) is referred to as pHMAAm. N-[3-(dimethylamino)propyl]acrylamide is referred to as DMAPAAm, (3-acrylamidopropyl)trimethylammonium chloride) is referred to as qNAAm, N-[tris(hydroxymethyl)methyl]acrylamide is referred to as TRIS-AAm, N,N-dimethylacrylamide is referred as DMAAm, 2-acrylamido-2-methyl-1-propanesulfonic acid is referred as AMPSA, 4-acryloylmorpholine is referred as AMP, N,N-diethylacrylamide is referred as DEAAm, acrylamide referred as AAm, sulfobetaine acrylamide is referred as SBAAm, carboxybetaine acrylamide is referred as CBAAm. Monomeric N-hydroxyethyl acrylate is referred to as HEA and poly(N-hydroxyethyl acrylate) is referred to as pHEA. Monomeric N-hydroxymethyl acrylate is referred to as HMA and poly(N-hydroxymethyl acrylate) is referred to as pHMA. N-hydroxysuccinimide is referred to as NHS and tris-[2-(dimethylamino)ethyl]amine is referred to as Me₆TREN. Tris(2-pyridylmethyl)amine is referred to as TPMA. Para-nitrophenyl butyrate is referred as pNPB. N-2-bromo-2-methylpropanoyl-3-alanine N′-oxysuccinimide ester is referred to as iBBr and N-2-bromo-2-propanoyl-β-alanine N′-oxysuccinimide ester is referred to as iPrBr. Phosphate buffered saline is referred to as PBS, (4-(2-hydroxyethyl)-1-piperazineethanesulfonic acid) is referred to as HEPES, borate buffered saline is referred to as BBS, and 2-(N-morpholino)ethanesulfonic acid is referred to as MES.

High-Throughput System

The present disclosure provides a high-throughput system capable of varying reaction conditions, monitoring reaction progress, and evaluating properties of synthesized enzyme-polymer conjugates. Enzyme-polymer conjugates are synthesized by conjugating polymers with specific functionality to an enzyme to form a complex that, ideally, combines the advantages of both the polymer and enzyme while negating weaknesses of each. The large number of different polymer types available, coupled with a wide range of conjugate functionality, makes it difficult to rapidly synthesize and optimize enzyme-polymer conjugates for a particular function or application. Additionally, it is possible to graft multiple types of polymers to the same enzyme, further expanding the number of possible reaction conditions to optimize. These polymers influence the final functionality of the enzyme-polymer conjugates, including chemical and thermal stability, size, catalytic activity, solubility, and pharmacokinetics.

While modification of enzymes may be achieved biologically by random mutation, or by cloning/expression, expression systems, biodiversity mining, site directed mutagenesis, or directed evolution, these methods often only yield incremental improvements, are difficult and expensive to scale, require long development times, and often only provide situational solutions. Current synthetic methods involve the slow process of varying and attempting to optimize each reaction parameter in turn. This may include optimization of type and concentration of polymer and enzyme, reaction time, temperature, and purification steps. This stepwise variation of reaction parameters leads to long periods of synthetic trial and error followed by even more laborious optimization of conditions.

The present disclosure offers a solution by using polymer-based enzyme engineering and high-throughput synthesis to rapidly screen reaction conditions and assess final enzyme-polymer conjugate functionality. An automated system of this nature may solve the arduous process of varying numerous parameters and permit exploration of enzyme-initiator and enzyme-polymer conjugate synthetic space in parallel, allowing for rapid generation of finished conjugates as well as large amounts of data concerning the effect of reaction conditions on final composition and functionality. Furthermore, a high-throughput system may be programmed with self-learning algorithms to take in data and results from first rounds of synthesis and act as a feedback loop to generate new conditions in an effort to Pareto optimize conjugate synthesis.

A system of this nature for simultaneously synthesizing a plurality of enzyme-polymer conjugates may comprise: (a) a plurality of reaction chambers configured to hold 1 to 1000 μL of fluid and to allow measurement of absorbance or fluorescence, by a spectrophotometer, of a enzyme-polymer conjugate contained in each reaction chamber in the plurality; (b) an automated device configured to deliver one or more of a reactant, solvent or catalyst to each reaction chamber in the plurality; (c) optionally, an agitation module configured to mix contents of each reaction chamber in the plurality; (d) a monitoring module configured to monitor progress of a reaction occurring in a reaction chamber in the plurality, wherein the monitoring module is in communication with a spectrophotometer configured to measure at least one of absorbance and fluorescence of the contents of at least one reaction chamber in the plurality; (e) a purification module in fluid communication with the plurality of reaction chambers, wherein the purification module is configured to separate an enzyme-polymer conjugate from other reaction mixture components, and wherein the other reaction mixture components comprise buffer, monomers and a catalyst; and (f) an evaluation module in visual communication with the plurality of reaction chambers, wherein the evaluation module is configured to assess one or more physical properties of an enzyme-polymer conjugate contained in each reaction chamber in the plurality.

The system may further comprise a photoirradiation module in visual communication with the plurality of reaction chambers, wherein the photoirradiation module is configured to initiate, by photoirradiation, a polymerization reaction in a reaction chamber in the plurality. The photoirradiation module may be configured to separately control the duration of photoirradiation for each of the plurality of reaction chambers. Optionally, the photoirradiation module is configured to separately control the intensity of photoirradiation for each of the plurality of reaction chambers. A system comprising a photoirradiation module may further comprise a temperature control module, such as a cooling module. The temperature control module may be configured to maintain the plurality of reaction chambers at a specific temperature, or within a specific temperature range. Optionally, the temperature control module comprises a fan. The fan may be placed under the photoirradiation module to provide cooling for the plurality of reaction chambers. If additional cooling is required, the temperature control module may further comprise a coolant. For example, a fan can be placed on top of a coolant to produce a cooler air stream directed toward the plurality of reaction chambers.

A central part of a high-throughput system of this nature is the ability to explore the synthetic space and variation of both enzyme-initiator and enzyme-polymer conjugates (FIG. 1). The synthesis of enzyme-polymer conjugates involves first attaching initiators to enzymes of interest to form enzyme-initiator conjugates. Enzyme-polymer conjugates may then be formed via polymerization of one or more monomers of interest. Determining and then optimizing advantageous properties of finished enzyme-polymer conjugates involves varying synthetic conditions for both attaching initiators to enzymes as well as grafting a polymer onto the enzyme via a polymerization reaction to form an enzyme-polymer conjugate. Both synthetic steps may alter the functionality of the resultant enzyme-polymer conjugates.

Enzymes

The first step in the synthesis of a biomolecule-polymer conjugate involves selecting a biomolecule of interest, such as a protein or enzyme. Proteins may be comprised of thousands to fewer than one hundred amino acid residues linked by peptide bonds, linearly and/or branched, and folded in three-dimensional configurations. The configuration of the protein determines structure and function. Exemplary proteins include chymotrypsin, lipase, nitrilase, bovine serum albumin, and antibody. In some embodiments, the biomolecule is an enzyme. Enzymes function as biological catalysts that may increase the rate of a biological reaction, such as by 10⁶ to 10¹⁴ fold. Most enzymes are reactive under mild physiological conditions. The configuration of an enzyme, and therefore, the position of available binding sites, contributes to the specificity and selectivity of the enzyme. Enzymes have an active binding site to receive and bind with a substrate, such as another protein, to form enzyme-substrate complexes. Upon binding, the enzyme catalyzes the relevant reaction to produce the end product of the catalyzed reaction. Enzymes interact with their substrates and targets by removing them from a solvent, binding, reacting and then returning products to solution. Exemplary classes of enzymes include esterases, lipases, transferases, oxidoreductases and proteases. In nature, there are complex interactions that dictate the final protein structure having its specific function. Controlled manipulation of the properties of a biomolecule, and in particular, an enzyme, may expand the scope of applications in which the biomolecule may be used, for example, in therapeutic applications. For example, an enzyme-polymer conjugate may retain the enzymatic activity of the native enzyme while having improved stability in a particular solvent, at a given pH, and/or at a specific temperature.

Polymers

Following selection of a biomolecule of interest, a polymer for covalent bonding is typically identified. Polymers may comprise thousands to fewer than five monomers linked by covalent bonds, linearly and/or branched, and may exist in folded three-dimensional structures, dependent on monomer structure and functionality. Exemplary classes of polymers may include polyacrylates, polyacrylamides, polymethacrylates, and polymethacrylamides. Optionally, the exemplary polymers are compounds of Formula (IV):

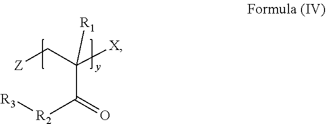

where X is a halogen or chain transfer agent; Z is an enzyme; R₁ is hydrogen or methyl; R₂ is ester or amide group; R₃ is a side chain that may comprise C₁₋₁₂ alkyl groups, hydroxyl groups, primary, secondary, tertiary or quaternary amine groups, anionic groups, cationic groups, zwitterionic groups or others; and y is an integer from 1 to 1000.

In some embodiments, the polymer of interest is a copolymer, comprising a combination of at least two or more different monomers. Copolymers may include linear block copolymers, linear alternating copolymers, linear periodic copolymers, linear random copolymers, linear gradient copolymers, branched copolymers, graft copolymers, or star copolymers. Polymers covalently bound to the biomolecule of interest may interact with themselves, each other, the surfaces of the biomolecule, the solvent, reaction reagents, ions in solution, or a combination thereof. These interactions may include, but are not limited to, electrostatic interactions, hydrogen bonding, hydrophobic interactions, functional group affinity, covalent bonding, or a combination thereof. Interactions of the covalently bound polymer may impart properties to the biomolecule of interest, including but not limited to, improved solubility, improved stability, improved affinity, improved activity, or a combination thereof.

Selection of the polymer of interest may depend on the desired biomolecule-polymer conjugate properties. In some embodiments, the desired biomolecule-polymer conjugate properties may include improved solubility in an alcohol solvent, or alcohol co-solvent, of interest. Preferred polymers may exhibit solubility in the alcohol of interest or may exhibit Hansen Solubility Parameters (HSP) compatible with the alcohol of interest. Polymer properties may be determined by the composition of monomers comprising the polymer. Exemplary monomers exhibiting alcohol solubility properties may include NIPAAm, HEAAm, HEA, HMAAm, and HMA, or monomers containing hydroxyl side chain functional groups. For example, polymers with desired alcohol solubility properties may comprise at least 10%, at least 20%, at least 30%, at least 40%, at least 50%, at least 60%, at least 70%, at least 80%, at least 90%, at least 95%, or 100% of one of these exemplary monomers, or a combination thereof.

In some embodiments, the desired biomolecule-polymer conjugate properties may include improved stability in an alcohol solvent, or alcohol cosolvent, of interest. Preferred polymers may impart stability through the occlusion of the alcohol of interest from the surface of the biomolecule, restricting reversible or irreversible denaturation. Occlusion of alcohol from the biomolecule surface may result from interaction of the polymer with the surface of a biomolecule through electrostatic interactions, hydrogen bonding, hydrophobic interactions, functional group affinity, covalent bonding, or a combination thereof. Exemplary polymers imparting stability to an alcohol of interest may include pNIPAAm, pCBAAm, pSBAAm, pDEAAm, pAMPSA, pAAm, zwitterionic polymers, polymers that interact with the surface of the biomolecule of interest, or a combination thereof. For example, biomolecule-polymer conjugates with desired alcohol stability properties may comprise at least 10%, at least 20%, at least 30%, at least 40%, at least 50%, at least 60%, at least 70%, at least 80%, at least 90%, at least 95%, or 100% of one of these exemplary polymers, or a combination thereof.

In some embodiments, the desired biomolecule-polymer conjugate properties may include improved affinity to an alcohol reagent or substrate of interest. Preferred polymers may exhibit affinity to the alcohol of interest. Exemplary polymers exhibiting alcohol substrate affinity may include pNIPAAm, pHEAAm, pHEA, pHMAAm, pHMA, or polymers containing hydroxyl side chain functional groups. For example, polymers with desired alcohol solubility properties may comprise at least 10%, at least 20%, at least 30%, at least 40%, at least 50%, at least 60%, at least 70%, at least 80%, at least 90%, at least 95%, or 100% of one of these exemplary polymers, or a combination thereof.

In some embodiments, the desired biomolecule-polymer conjugate properties may include improved activity to an alcohol reagent or substrate of interest. Preferred polymers may exhibit affinity to the alcohol of interest, improved solubility in an alcohol of interest, or improved stability in an alcohol of interest. Exemplary polymers exhibiting alcohol substrate affinity may include pNIPAAm, pHEAAm, pHEA, pHMAAm, pHMA, pNIPAAm, pCBAAm, pSBAAm, pDEAAm, pAMPSA, pAAm, zwitterionic polymers, polymers that interact with the surface of the biomolecule of interest, polymers containing hydroxyl side chain functional groups, or a combination thereof. For example, polymers with desired alcohol solubility properties may comprise at least 10%, at least 20%, at least 30%, at least 40%, at least 50%, at least 60%, at least 70%, at least 80%, at least 90%, at least 95%, or 100% of one of these exemplary polymers, or a combination thereof.

Polymerization Methods

To broaden the application of proteins for industrial applications, research has been focused on methods to generate stable, selective and productive proteins and enzymes. Modern engineered proteins can accept a variety of substrates and transform them into novel materials, high-value chemicals, and renewable biofuels. Polymer-based protein engineering has emerged as a powerful tool in a protein scientists' toolbox to improve protein properties to facilitate their efficient application in therapeutic, diagnostic and catalytic industries. Covalent attachment of a water-soluble polymer to a protein may improve physical stability, proteolytic stability, and pharmacokinetics in pharmaceutical applications. The same approach has been applied in biocatalysis, where polymer-grafted enzymes displayed increased solution and thermal stability, as well as improved performance in non-aqueous solvents.

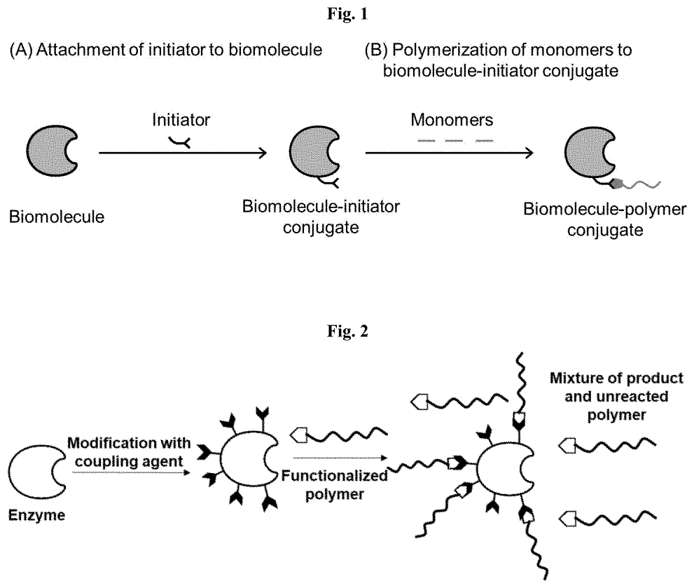

However, broad commercial utilization of protein-polymer conjugates is immature: they are primarily utilized in the pharmaceutical industry. Preparation of protein-polymer conjugates can be expensive and the efficacy of the final product can depend on the molecular weight and dispersity of the polymer, as well as the attachment point of the polymer onto the enzyme. The current commercial method for therapeutic protein-polymer conjugate synthesis is a “grafting to” approach (FIG. 2). The “grafting to” approach links a well-defined, pre-formed polymer with a reactive chain-end to a corresponding functionalized protein. This approach has a major drawback in that not all available attachment sites can be functionalized due to the steric hindrance between polymer chains, leading to an inhomogeneous product with inconsistent results. Furthermore, purification of free polymer, especially of higher molecular weight, from the conjugated product is difficult due to their similarly large molecular weights and requires the use of expensive size-exclusion chromatography. The “grafting from” approach starts with modification of a protein with polymer initiator functionalities, which can be further extended by polymerization (FIG. 1). Reversible deactivation radical polymerization (RDRP) methods such as ATRP and RAFT can be utilized to grow polymers from the protein surface. This approach leads to high yields of well-defined enzyme-polymer conjugate and significantly simpler purification, because the enzyme-polymer conjugate needs only to be separated from small-molecule monomers. Therefore, enzyme-polymer conjugates prepared by the “grafting from” method can be produced with reduced cost and more consistent batch-to-batch composition.

The essence of control over polymerization via available RDRP methods, previously known as “living” or controlled radical polymerization (CRP), lies in the development of procedures where most of the radical precursors are present in their dormant state and only a small fraction of potential radicals can propagate at any instance. Currently, several RDRP methods have been reported and utilized including nitroxide mediated polymerization (NMP), reversible addition-fragmentation chain transfer (RAFT) polymerization, atom transfer radical polymerization (ATRP) and some others. Among them, ATRP gained popularity since its discovery in 1995 and currently remains the most often utilized RDRP technique. The main advantages of ATRP include commercial availability of almost all polymerization components, such as initiators and catalysts, use of catalytic amounts of metal complexes, ease of chain end modification, the ability to polymerize a wide range of radically copolymerizable monomers and incorporate macromolecules prepared by other polymerization procedures.

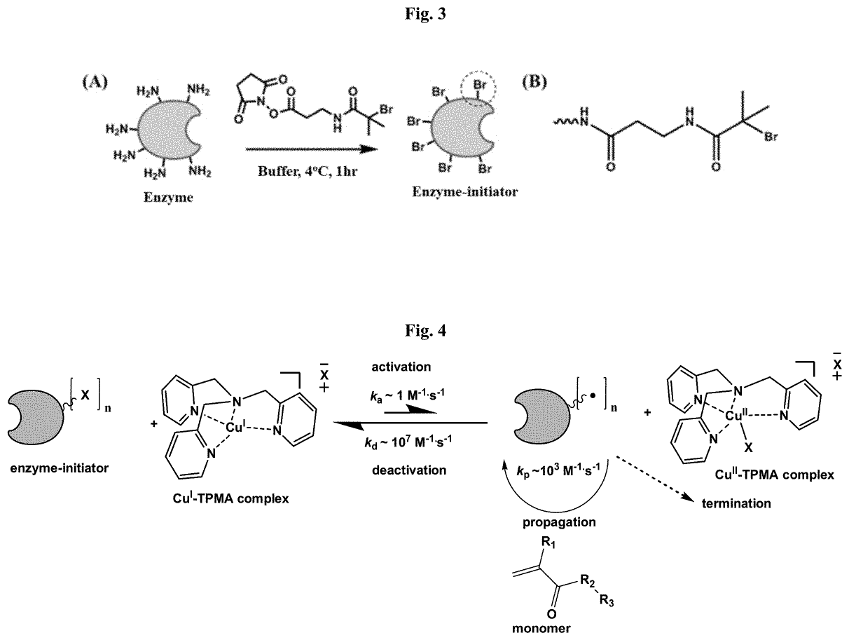

To date, multiple chemistries have been developed for the modification of proteins with polymerization initiation moieties. Lysine residues are among the most commonly modified functionalities, which can be achieved without denaturation of a protein. This approach would typically result in multiple initiating sites on the surface of a protein. This method relies on a natural structure of a protein and does not require genetic engineering methods. In the case of labelling of an enzyme with ATRP initiating moieties, the typical reaction condition includes mixture of an enzyme in a selected buffer to which ATRP initiator with NETS-functionality is added at a selected ratio to lysine residues of the enzyme (FIG. 3). Buffer type, pH and ratio of enzyme-reactive ATRP initiator can be varied to control how many lysine residues are modified with ATRP initiator moiety. Upon completion of the reaction the enzyme-initiator can be purified either by dialysis or ultrafiltration methods to remove reaction byproducts. The labelling efficiency of the enzyme with ATRP initiators moieties can be determined by fluorescence-based methods such as fluorescamine assay, or by mass spectrometry methods such as ESI-MS or MALDI-TOF. The retained enzymatic activity of enzyme-initiator can be determined by suitable enzymatic assay.

During an ATRP reaction, the carbon-halogen bond in an alkyl halide is reversibly cleaved by a reaction with a transition metal complex in its lower oxidation state, which results in the formation of a radical and a metal complex in its higher oxidation state (FIG. 4). The resultant carbon radical can propagate, terminate or react with the metal-halide complex to reform a dormant species. Specific conditions are selected such that active radicals are rapidly deactivated, making the dormant state the majority species. Lower oxidation state transition metal complex can be added directly to the reaction mixture to initiate polymerization, or it can be generated in situ by reducing lower oxidation state transition metal complex by means of chemical reducing agent, electrochemical reaction, photo irradiation, and other methods. Due to the high fraction of dormant chains, termination usually does not exceed 1-10%, depending on conditions. In this way, ATRP, similar to other living polymerization methods, allows for precise control over macromolecular composition, architecture and functionality.

When properly formed, enzyme-polymer conjugates display the same specificity and reactivity as native enzymes, but with additional polymer dependent properties. Enhanced stability under extreme conditions (i.e., elevated temperature, non-neutral pH, and organic solvents) can be applied to an enzyme via polymerization, without the time and effort of traditional protein engineering methods.

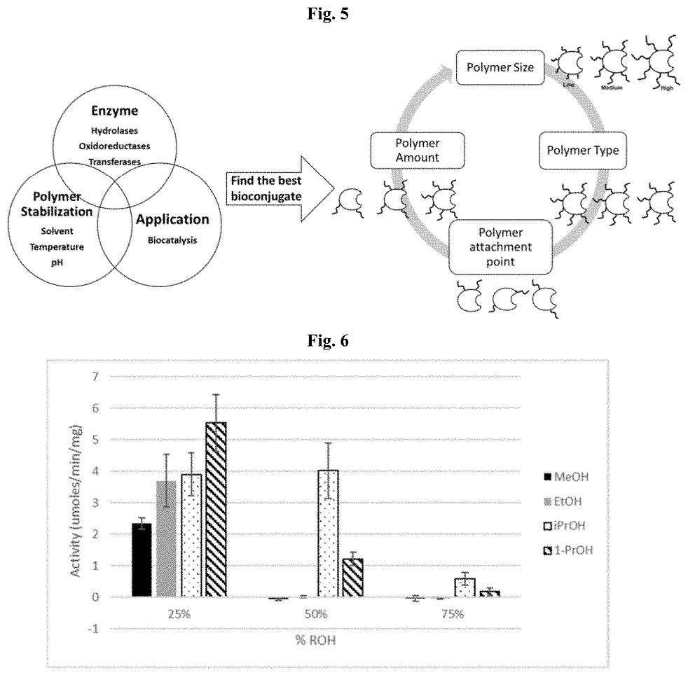

Another advantage of ATRP for conjugating polymers to enzymes is its high versatility and tunability. Polymer size, type, amount, and attachment point can be tuned to optimize conjugates for particular applications (FIG. 5). Additionally, polymerization conditions can be controlled to a high degree, optimizing final product quality, yield, and performance. Polymer conjugation can thus be carefully customized for each specific enzyme and application. Such versatility and tunability generates a much broader search space for optimization, making the rational design process lengthier and more limited. Thus, application of high-throughput combinatorial enzyme-polymer conjugate synthesis and screening platform allows discovery of optimal enzyme-polymer conjugate formulations for specified applications. High-throughput combinatorial protein-polymer conjugate synthesis and characterization can drastically accelerate the identification of enzyme-polymer conjugates with targeted properties.

Alcohol Stable Enzyme-Polymer Conjugates

Increased tolerance to alcohols is a highly desired stabilization effect because short-chain alcohols are often industrially utilized as reagents and co-solvents. Polar organic solvents such as methanol usually cause inactivation and unfolding of proteins due to a combination of stripping of water from the protein surface, and collapse of buried hydrophobic regions of the protein due to the destabilizing effect of aliphatic alcohols on tertiary interactions. For example, lipase type A from Candida antarctica (CALA) loses all of its activity upon incubation for an hour in alcohol mixtures with buffer, where alcohol content is over 25% by volume (FIG. 6). Thus, alcohols hamper the activity of enzymes when used at concentrations beneficial for biocatalytic reactions (over 25% by volume in aqueous buffer). Increased stability in alcohol would allow for increased alcohol concentration in a reaction, which would allow for higher conversion and yield.

It has been reported that some level of alcohol stabilization can be achieved by treatment of enzymes with salts, sugars and compounds with multiple hydroxyl groups. It is hypothesized that such pretreatment can improve enzyme stability due to water distribution on the enzyme surface, reducing stripping of the water from the enzyme surface. However, such pretreatment did not produce stabilizing effect beyond 20 minutes. Most biocatalytic reactions are performed for at least 24 hours, and thus development of enzymes that are stable for prolonged periods of time is important for further advancement of industrial biocatalysis. Other reported approaches include encapsulation, where the enzyme surface is crosslinked with a polymer shell to retain the enzyme folded structure. For this approach, only a short stabilization effect of 10 minutes was demonstrated.

Genetic engineering methods were shown to be more efficient for generation of more stable and active enzymes. Site-directed mutagenesis and directed evolution are among the methods that allow manipulation of the protein amino acid sequence for generating new proteins which exhibit broader substrate range and higher efficiency. However, a limited number of cases were reported that showcased significant improvements in stability in organic solvents where the volume fraction of organic solvent is at least 20 percent. For the stability of enzymes in alcohols, one of the most successful examples include Proteus mirabilis lipase which underwent 4 rounds of directed evolution to achieve stability in 50% by volume methanol solution. Genetic engineering approaches can be expensive and require long product development cycles. Desirable protein properties such as thermal and solvent tolerance are mostly dependent on global protein folding, and thus it is difficult to attain such robust proteins through single mutation or structural domain alteration.

Another approach to stabilize enzymes is to immobilize them on a solid support, which also provides an opportunity for the recycling of the catalyst thereby bringing costs down, as well as simplifying downstream processing and product separation. For example, ketoreductase was covalently immobilized on an epoxide-functionalized support, which provided an opportunity to shift operation conditions from 30% of isopropanol in buffer to 90% of isopropanol in buffer crucial for reagents and product solubility. However, while some immobilized enzymes can be successfully utilized in industrial processes, others denature upon immobilization. Additionally, immobilization of enzymes negatively influences reaction kinetics and efficiency, which necessitates the need for a novel approach towards improving the stability and lifetime of enzymatic catalysts.

It was previously demonstrated that covalent modification of enzymes with synthetic polymers can provide stability and activity to an enzyme in organic solvents. For example, pAMP grown from a lipase provided stability and 21-fold increased activity to this enzyme in ionic liquid. Furthermore, it was shown that polymer like pDMAEMA when covalently attached to chymotrypsin can provide activity and stability to this enzyme in acetonitrile and dichloromethane. But addition of n-propanol in concentrations higher than 10% by volume resulted in enzyme inactivation. Thus, the stabilization of enzymes against the denaturing effect of polar alcohols through polymer conjugation has not been reported before.

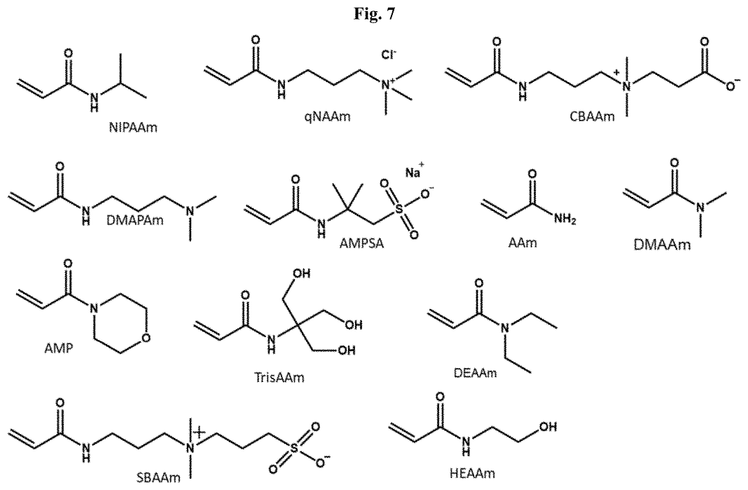

Herein, we have generated and screened almost 500 enzyme-polymer conjugates, where polymer type, size and coverage was varied to find samples with higher alcohol stability in comparison to the native, unmodified enzyme. Enzyme stability in alcohol mixtures was used to isolate samples stable to denaturation in the presence of alcohols and correlated with effect of polymer modification on enzymatic kinetics. Twelve different polymer types (FIG. 7) were tested if they conferred enhanced stability to an enzyme in partial-alcohol solvents, and which polymerization conditions resulted in the highest quality enzyme-polymer conjugates. Purified and characterized enzyme-polymer conjugates were tested for their retained enzymatic activity utilizing an aqueous hydrolysis reaction where release of the product was detected by UV-Vis spectrophotometry. Enzyme-polymer conjugates with at least 30% of residual enzymatic activity under standard testing conditions were further tested for the stability in the presence of alcohols.

Enzyme-polymer conjugates were subjected to incubation in the presence of selected alcohols at varied ratios with buffer. Incubation time was 24 hr. Upon completion of incubation, enzyme-polymer conjugates were tested for enzymatic activity retention utilizing aqueous hydrolysis reaction. Samples with high activity retention of at least 20% in comparison to native enzyme were accounted for stabilized polymer effect.

Enzyme-polymer conjugate activity was also measured in the presence of selected alcohols at varied ratios with buffer. The end point at 5 min was diluted with aqueous buffer to ensure absorption detection of the product of the reaction and measured by UV-Vis spectrophotometry. Samples with retained enzymatic activity higher by at least 20% in comparison to native enzyme were selected for further optimization. The successful candidates of the enzyme-polymer conjugates retained at least 20% activity in an aqueous solution comprising at least 30% of a C₁₋₃ alcohol relative to the activity of the native enzyme in an aqueous solution comprising less than 5% of the C₁₋₃ alcohol, such as at least 35%, at least 40%, at least 45%, at least 50%, at least 55%, at least 60%, at least 65%, at least 70%, at least 75%, at least 80%, at least 85%, at least 90%, or at least 95% of the activity relative to the native enzyme. The successful candidates of the enzyme-polymer conjugates retained at least 20% activity in C₁₋₃ alcohol and water mixture comprising at least 30%, at least 35%, at least 40%, at least 45%, at least 50%, at least 55%, at least 60%, at least 65%, at least 70%, at least 75%, at least 80%, at least 85%, at least 90%, or at least 95% of a C₁₋₃ alcohol relative to the aqueous component.

In certain examples, the density of polymer covalently bound to the biomolecule surface is dependent on the number of accessible lysine residues present on the biomolecule surface. The lysine residues are modified with ATRP initiator, allowing for growth of polymer from the biomolecule surface. Therefore, the biomolecule surface contains a plurality of sites for polymer growth, wherein that plurality is an integer between 1-100. Different degrees of modification of the biomolecule may be achieved by varying the reaction conditions, including, for example, reaction pH, reaction temperature, buffer type, additives (e.g., glycerol or propylene glycol), reaction time, identity of the biomolecule, concentration of the biomolecule, equivalents of the biomolecule, identity of the controlled radical polymerization initiator, concentration of the controlled radical polymerization initiator, and equivalents of the controlled radical polymerization initiator. The degree to which the biomolecule is modified by the initiator at this stage controls the ultimate polymer coverage of the biomolecule surface in the second stage when biomolecule-polymer conjugates are synthesized. The reaction progress can be monitored spectrophotometrically. The efficiency of the biomolecule-initiator conjugate reaction can be assessed using a fluorescamine assay, which allows for the quantification of modified amino groups on the biomolecule. Activity of the biomolecule, such as enzymatic activity, can be assessed on a model reaction utilizing the biomolecule-initiator conjugate. Biomolecule-initiator conjugates that exhibit significantly reduced activity may be discarded prior to grafting a polymer to the conjugates. Optionally, activity of biomolecule-initiator conjugates is assessed, and only biomolecule-initiator conjugates that retain at least 30%, at least 40%, at least 50%, at least 60%, at least 70%, at least 80%, at least 90%, or at least 100% of the original biomolecule activity are reacted with monomers under controlled radical polymerization conditions suitable for forming a plurality of biomolecule-polymer conjugates.

The ratio of amount of initiator to biomolecule during biomolecule-initiator synthesis plays a large role in the properties of biomolecule-polymer conjugates. Using a larger quantity of initiator may lead to higher initiator density on the surface of the biomolecule and better control over the eventual size of the synthesized conjugate while utilizing the “grafting from” conjugation technique. In some embodiments, biomolecule-initiator conjugates are classified into “minimal”, “partial”, or “maximum” densities, based on the degree of modification. “Minimal” density may include biomolecule-initiator densities <33% of maximum density, “partial” density may include biomolecule-initiator densities 33-67% of maximum density, and “maximum” density may include biomolecule-initiator densities >67% of maximum density. The “maximum” density may be defined as “total number of lysine residues” plus “one”, where “one” comes from the amino-group at the N-terminus of an enzyme.

Polymers may be covalently bound to the surface of the biomolecule through the ATRP initiator. The linkages attaching polymer to the biomolecule may be, but are not limited to, amide bonds, ester bonds, disulfide bonds, or triazole bonds. The polymer may be grown directly from a biomolecule-initiator conjugate, or grown from an initiator that is attached to the biomolecule through a linker, such as, but not limited to, poly(ethylene glycol), C₁₋₂₀ alkyl chains, polypeptides, or aryl groups. Different degrees of modification of the biomolecule may be achieved by varying the reaction conditions, including, for example, reaction pH, reaction temperature, buffer type, additives (e.g., glycerol or propylene glycol), reaction time, identity of the biomolecule, concentration of the biomolecule, equivalents of the biomolecule, identity of the monomer, concentration of the monomer, equivalents of the monomer, identity of the catalyst, concentration of the catalyst, equivalents of the catalyst, photoirradiation intensity, and photoirradiation duration.

Methods of Use

The claimed enzyme-polymer conjugates have broad applicability across the field of industrial biocatalysis. In many biocatalytic reactions, alcohols are used as reagents, solvents, co-solvents, or a combination thereof. As such, improved enzymatic stability in alcohol containing solutions, imparted by covalent bonding of polymers, is of significant importance. In certain examples, polymers imparting enzymatic stability to methanol are of use in the field of biodiesel production. Methanol is used as a reagent in the production of biodiesel from biomass oils, such as soybean oil. Enzymatic biodiesel production is limited by current enzyme stability in the presence of methanol. Enzymatic biodiesel production is often performed by lipases and esterases.

In another example, polymers imparting enzymatic stability to short alkyl chain alcohols are of use in the field of active pharmaceutical ingredient (API) synthesis. API synthesis often requires the addition of alcohol solvents or co-solvents for the solubilization of reagents or products, as such enzymatic biocatalysis of APIs is limited by enzyme alcohol stability. Examples of classes of enzymes used for the synthesis of APIs include, but is not limited to, lipases, amidases, transaminases, ketoreductases, and cytochromes.

Monitoring, Evaluation, and Screening

A major advantage in utilizing automated synthetic systems is the integration of in situ reaction monitoring systems to assess reaction progress and/or purity of synthesized materials. A common term for these methods of testing quality as a reaction proceeds is process analytical technology (PAT). Commonly employed in pharmaceutical manufacturing systems, these are automated and integrated technologies, ranging from organic synthesis to spectrometric and chromatographic systems, which are used to assess quality of a final product. PAT methods of the present disclosure typically comprise data analysis, process analytical tools, process monitoring, and continuous feedback. Methods for real-time analysis of various steps may include FTIR spectroscopy for reaction analysis; NIR spectroscopy to measure product uniformity; and HPLC, GC, NMR spectroscopy, DLS, and MS for reaction analysis and product identity. These techniques may be applied to the synthesis of biomolecule-polymer conjugates, with particular interest being paid to initiator synthesis, attachment of initiators to biomolecules, and tracking of reaction progress, including concentration of monomers during the synthesis of biomolecule-polymer conjugates. For example, proton NMR can be used to measure conversion, and molecular weight and dispersity can be measured by aqueous GPC. Gel electrophoresis of biomolecule-polymer conjugates can reveal the amount of unconjugated biomolecule present in a reaction mixture to assess reaction efficiency and can further be used to assess the size of biomolecule-polymer conjugates. Additionally, DLS analysis of biomolecule-polymer conjugates provides conjugate hydrodynamic radii, allowing for size estimation, aggregation monitoring, and even temperature-responsive changes in physical properties, such as lower-critical solution temperature (LCST) characteristics imparted by the covalently bound polymer.

PAT techniques may also be used in the purification of materials. Purification of conjugates typically comprises increasing the number of biomolecule-initiator or biomolecule-polymer conjugates relative to undesired side products. One aspect of the purification described herein comprises a method of simultaneously isolating a plurality of bioconjugates from a plurality of reaction mixtures. In some embodiments, the method comprising simultaneously passing a plurality of reaction mixtures comprising a plurality of bioconjugates through a plurality of ultrafiltration membranes, wherein the bioconjugates are retained above the membranes, wherein the bioconjugates comprise a biomolecule conjugated to a controlled radical polymerization initiator or a biomolecule conjugated to a synthetic polymer, and wherein each reaction mixture in the plurality is independently purified. The ultrafiltration may be vacuum-assisted or centrifugal force assisted. These purification steps and methods may be incorporated into the high-throughput automated system by utilizing the membranes in the plurality of reaction chambers. The membranes may be ultrafiltration membranes that allow small molecules such as water to pass through, but retain larger molecules such as proteins or other biomolecules. This gives the advantage of washing away unattached initiators or monomers from the biomolecule-initiator and biomolecule-polymer conjugates, respectively. An additional advantage of using an automated system is that each reaction chamber may be individually addressable and the ultrafiltration membranes may be configured to allow continuous fluid delivery through the membranes, such as during purification.

Purification of the bioconjugates may be aided by the attachment or immobilization of a biomolecule or a biomolecule-initiator conjugate to a reaction chamber. Flowing fluid through a chamber with an immobilized biomolecule or biomolecule-initiator conjugate may assist in purification, as excess initiators or monomers will be filtered out of the chamber while only the biomolecule-initiator conjugate or biomolecule-polymer conjugate remains. Immobilization methods vary largely with immobilization surface, biomolecule properties, and the desired functionality of the final biomolecule-polymer conjugate. Proteins and other biomolecules may be attached to a surface by one of several different mechanisms. For example, a biomolecule may be attached via passive adsorption, in which the attachment is via hydrophobic interactions or hydrophobic/ionic interactions between the biomolecules and the surface. Covalent immobilization may be used to immobilize a biomolecule to a surface. For example, amine-based covalent linking may be used, utilizing lysine residues on the surface of a biomolecule. Any of these immobilization techniques may be employed during synthesis of biomolecule-polymer conjugates, and it may be found that a specific immobilization technique helps speed up the purification or isolation of biomolecule-initiator and/or biomolecule-polymer conjugates.

Biomolecule-initiator and/or biomolecule-polymer conjugates may be evaluated and/or screened for one or more properties. In some embodiments, the evaluating and/or screening is conducted after purification of the conjugates, though purification may not be required. Utilizing a high-throughput system, reaction chambers can be configured such that absorbance or fluorescence of a reaction mixture or a purified conjugate can be accurately measured by a spectrophotometer. Other evaluation steps may comprise measuring one or more of ultraviolet-visible spectroscopy, fluorescence spectroscopy, near-infrared spectroscopy, and size assessment. Activity, such as enzymatic activity, of biomolecule-initiator and/or biomolecule-polymer conjugates may be assessed under ideal working conditions, then optionally under stress conditions—such as high temperature, extreme pH or various solvent mixtures—to identify conjugates that exhibit improved activity and/or stability relative to the native biomolecule. In some embodiments, the activity of the enzyme of interest can be measured by monitoring hydrolysis of a reporter molecule, such as p-nitrophenyl butyrate (pNPB). Hydrolysis of pNPB produces p-nitrophenol, a colorimetric molecule, the production of which can be spectroscopically monitored. These varied evaluation techniques help identify and assess desirable properties of conjugates synthesized according to the methods described herein, such as density of initiators on the biomolecule surface, catalytic activity, stability in a particular media and/or condition, as well as degree of biomolecule modification and polymerization. In certain embodiments, the density of initiators on the biomolecule surface can be determined spectroscopically using an amine-responsive fluorophore, such as fluorescamine, which becomes fluorescent upon binding to primary amines, such as lysine residues present on the surface of a biomolecule of interest. Comparison of available surface lysine residues between biomolecule-initiator conjugates and unmodified biomolecule allows for calculation of initiator density on the surface of the biomolecule of interest. The advantage of using a high-throughput system lies in the fact that conjugates that display one or more properties deemed to be advantageous for a particular application may be easily isolated and similar synthetic space may be explored for biomolecule-initiator and biomolecule-polymer conjugate optimization.

Screening for optimized conditions for polymerization reactions is especially difficult as these reactions are often highly dependent on reaction kinetics. Thus, fast and comparable screening of different polymerization parameters under similar conditions is useful in evaluating many different polymerization conditions. The ability to rapidly screen conditions in parallel allows for direct comparison of different reaction conditions and helps eliminate handling errors, which often affects the results of a kinetic experiment. Automated high-throughput systems allow one to quickly focus on a particularly robust set of reaction conditions and produce biomolecule-initiator or biomolecule-polymer conjugates with a specific function while eliminating reaction conditions that produce conjugates which are unstable or unsuitable for a given application. Small changes in reaction conditions may produce conjugates with vastly different functionality. Thus, parallel screening of reaction conditions is a valuable addition to the synthetic tools available in production of biomolecule-initiator and biomolecule-polymer conjugates.

While parallel screening is an extremely useful tool to explore synthetic conditions and assess function of conjugates, automated feedback loops are useful in generating new synthetic conditions and preparing libraries of biomolecule-initiator and biomolecule-polymer conjugates. These libraries can be narrowly defined for a particular functionality after synthesis of initial conjugates. By screening these initial results and identifying hits for a useful property of interest, a smaller area of synthetic space can be explored and conditions that would not produce usable conjugates can be discarded. This feedback loop is an integral part of rapid parallel screening of conditions that may not initially seem obvious. Additionally, this feedback-loop system, which may comprise a self-learning algorithm, can be repeated for several generations of synthetic optimization for Pareto optimization of biomolecule-polymer conjugates for a given application.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

Lipases are a class of enzymes that have many industrial applications. For example, lipases are used in laundry detergents and food processing, but also for generation of biofuels and high-value added pharmaceutical compounds. In preparation for growing polymers from the surface of enzymes, primary amine groups on lysine amino acids and the N-terminus are modified with an ATRP initiating molecule. The efficacy of modification is dependent on initiator and amine group concentrations, as well as reaction pH. Disruption of surface charges from blocking lysine residues with an initiator can, in some cases, impact the enzymatic activity. Conditions for modifying lipases were investigated, and the effect of the modifications on enzyme activity was monitored using a spectroscopic para-nitrophenyl butyrate (pNPB) hydrolysis assay. To demonstrate the versatility of enzyme-polymer conjugation, lipases from Candida antarctica (Lipase Type B and Type A), Thermomyces lanuginosus, and Candida rugosa were selected for modification. The chosen lipases differ by molecular weight and number of amine sites (Table 1).

TABLE 1
Properties of the selected lipases.

| | Lipase | | | Lipase |
|---|---|---|---|---|
| | Type B | Lipase | Lipase | Type A |
| | from | from | from | from |
| | Candida | Thermomyces | Candida | Candida |
| Enzyme | antarctica | lanuginosus | rugosa | antarctica |
| Total residues | 317 | 269 | 534 | 440 |
| Lys residues | 9 | 7 | 20 | 19 |

High-throughput combinatorial enzyme-polymer conjugate synthesis and characterization was achieved by subjecting the enzyme to step-wise modification, purification, and characterization. In the first stage, the enzyme was modified with polymerization initiating moieties and the effect of modification on enzyme performance was tested. In one example, the controlled radical polymerization initiator is an NHS-functionalized amide containing ATRP initiator, such as N-2-bromo-2-methylpropanoyl-β-alanine N′-oxysuccinimide ester:

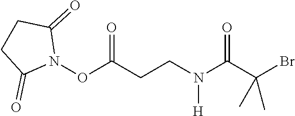 [SMILES: CC(C)(Br)C(=O)NCCC(=O)ON1C(=O)CCC1=O]

Different degrees of modification were achieved by varying reaction conditions (Table 2), resulting in control over final polymer coverage of the enzyme surface. The synthesized enzyme-initiators were then purified by high-throughput vacuum assisted ultrafiltration using a multi-well plate vacuum manifold utilizing multi-well filter plates with 10 KDa or 30 KDa molecular weight cutoff membranes. Enzymatic activity of enzyme-initiators was analyzed in a model reaction, where reaction progress was monitored spectrophotometrically, a technique suitable for high-throughput characterization. The outcome of this stage was a library of enzymes labelled with polymerization initiator (enzyme-initiators) where reaction conditions were correlated with the labelling density and retained enzymatic activity.

TABLE 2
Example of enzyme-initiator synthesis
reaction conditions using Lipase CAL A.

| | | Buffer | [Initiator] | Initiator/ |
|---|---|---|---|---|
| Sample # | Buffer | pH | (mM) | Enzyme |
| 1 | MES | 6 | 3.4 | 7.4 |
| 2 | MES | 6 | 12.9 | 8.6 |
| 3 | MES | 6 | 22.9 | 10.0 |
| 4 | PBS | 7.4 | 3.4 | 14.2 |
| 5 | PBS | 7.4 | 9.5 | 15.4 |
| 6 | PBS | 7.4 | 9.8 | 15.2 |
| 7 | PBS | 7.4 | 18.2 | 15.4 |
| 8 | PBS | 7.4 | 18.2 | 15.3 |
| 9 | PBS | 7.4 | 23.8 | 15.3 |
| 10 | HEPES | 8 | 3.4 | 17.4 |
| 11 | HEPES | 8 | 13.8 | 18.5 |
| 12 | HEPES | 8 | 23.8 | 18.6 |

### Example 2

Polymerization conditions were optimized and polymer configurations that grant lipase stability in partial-alcohol environments were screened in a high throughput approach. Nearly 500 unique bioconjugates were generated, and a polymer granting increased lipase stability in partial-alcohol environments was identified.

An Optimal Design of Experiment (ODE) was used to conduct a comprehensive response surface experiment with all possible variables for acrylamide (AAm) polymerization onto a model protein, Bovine Serum Albumin (BSA). Target properties of the resultant protein-polymer conjugate include a polydispersity index (PDI)<1.5, and identification of factors (Table 3) that determine polymer molecular weight and bio-conjugate yield from modified enzyme.

TABLE 3
Factors used in ODE of high-throughput experiment of polymerization
of AAm onto BSA with their type and investigated levels.

| Factor | Type | Levels | |
|---|---|---|---|
| Ligand Type | Categorical | TPMA, Me6TREN | |
| [Catalytic | Continuous | 0.3-10 | mM |
| Complex] | | | |
| Light Intensity | Discrete Numeric | 15, 30 | mW |
| (mW) | | | |
| [Triethylamine] | Continuous | 0-100 | mM |
| Buffer Type | Categorical | PBS, HEPES | |
| [Monomer] | Continuous | 50-400 | mM |
| Reaction Time | Discrete Numeric | 30, 60, 90, 120, | |
| | | 150, 180, 210, and | |
| | | 240 minutes | |
| [NaCl] | Continuous | 0-100 | mM |

| | | |
|---|---|---|
| Initiator Type | Categorical | iBBr, iPrBr |

The type of ligand forming the catalytic complex, as well as the overall concentration of the complex itself, influences the uniformity of chain growth. Reduction of the catalytic complex for propagating chain growth was accomplished by varying triethylamine concentration and blue light intensity. The free chloride ion in the phosphate-buffered saline is known to induce halogen exchange with chain ends, affecting polymer formation, therefore an alternative halogen-free buffer, HEPES, was also included in the trial. For the same reason, the concentration of sodium chloride was varied from 0 to 100 mM. The concentration of monomer relative to the concentration of initiation sites in each reaction determined the theoretical degree of polymerization (DP), and thus the maximum possible molecular weight of each polymer chain. The concentration of initiation sites was kept constant at 1 mM.

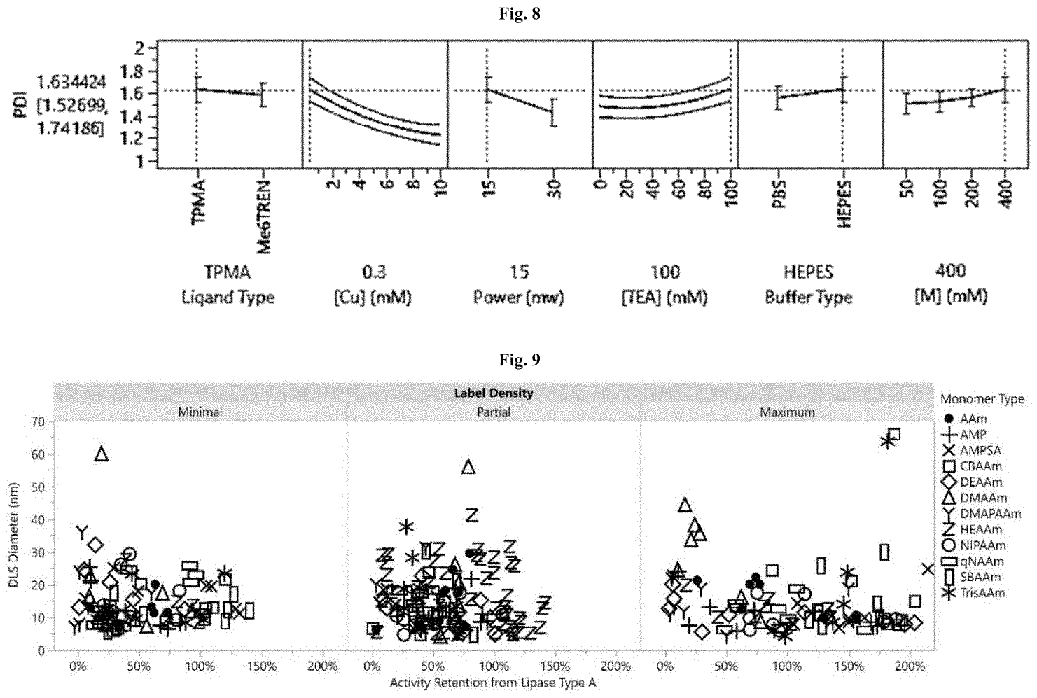

Determining a statistically-significant evaluation of influence for each of the experimental factors on each of the response variables required 189 bio-conjugates. Following GPC analysis of each bio-conjugate, a separate model per response variable was created. Models for prediction of PDI and percent yield both achieved a P-value of <0.001 and provided valuable insight into which factor levels had the most significant influence over good polymer formation (Table 4, FIG. 8).

TABLE 4
Model parameter estimates for predicting PDI and % Yield, or initiation
efficiency, produced from the high-throughput response-surface
experiment of bioconjugation between acrylamide and BSA. Ranked
in descending order of most influential, with associated parameter
estimates used for predicting response variables.

| PDI | | % Yield | |
|---|---|---|---|
| | Esti- | | Esti- |
| Parameter | mate | Parameter | mate |
| Power (mW) | −0.09 | Ligand Type | −0.30 |
| Ligand Type * [Catalytic | −0.07 | Ligand Type * [Catalytic | −0.14 |
| Complex] (mM) | | Complex] (mM) | |
| Ligand Type | −0.06 | [M] (mM) | 0.14 |
| [M] (mM) | 0.06 | [NaCl] (mM) * [M] (mM) | 0.14 |
| [TEA] (mM) | 0.04 | Reaction Time (min) * [TEA] | −0.11 |
| | | (mM) * Buffer Type | |

The results of this high-throughput screening experiment provided factors that could remain fixed in future polymerizations: using 15 mW light source strength, iBBr initiator type, and PBS buffer. Additionally, factors that could be varied to optimize conjugation for each lipase target were identified. The developed conditions were further applied to generate a library of enzyme-polymer conjugates.

### Example 3

The library of enzyme-initiators then entered the second stage, where polymers were grafted directly from enzymes utilizing optimized conditions. Constant polymerization conditions previously identified included light source power, enzyme-initiator synthesis with iBBr, copper (II) source, and polymerization in PBS, variable reaction conditions are shown in Table 5. This experiment utilized an oxygen-tolerant ATRP method where photoirradiation can induce polymerization (photoATRP). Such photoATRP system is oxygen tolerant, eliminating the need for an inert atmosphere, and can be performed in 96 well plates using a non-destructive blue light source. The high-throughput growth of polymers from an enzyme using a straightforward experimental system and affordable instrumentation was demonstrated in this experiment. Following polymerization, enzyme-polymer conjugates were purified by centrifugal ultrafiltration with 10 KDa and 30 KDa molecular weight cutoff ultrafiltration membranes, removing all small molecules including leftover monomer and catalyst. Enzymatic activity of the purified samples was assessed under ideal reaction conditions (30° C., 250 nM lipase, 160 μM pNPB in 100 μL pH 8 TrisHCl, 0.001% gum Arabic and 0.004% Triton X-100). Enzymatic activity was calculated from the initial rate of product formation as a slope of initial linear region. Samples were then subjected to stressed conditions to identify better performing polymer-modified enzymes. Stressed conditions included 24-hour incubation of enzyme-polymer conjugates at 30° C. in alcohol-buffer solutions. Samples were incubated with methanol, ethanol, n-propanol, and isopropanol at 0, 25, 50, and 75% (v/v) in PBS. One benefit of this combinatorial photo-ATRP system is in rapidly generating a large library of enzyme-polymer conjugates with varied polymer properties (i.e., coverage density, type, size, composition, and architecture), and matching it to enzymatic performance, identifying the influence of polymer modification on enzyme properties. Additionally, application of this process in an iterative manner provides the opportunity to combine discovered properties (e.g., bioconjugates with both temperature- and pH-stability) to optimize enzymatic performance for a chosen application.

TABLE 5
ATRP polymerization reaction condition matrix.

| | | | | | Variable |
|---|---|---|---|---|---|
| | Constant | | Variable | | type |
| Labeling | | | 4.1, 6.6, 7.9, | Br/Lipase | discrete |
| Density | | | 8.2, 14.6, 15.7, | | |
| | | | 17.3, 18.6 | | |
| [TEA] | | | 0-100 | mM | continuous |
| [Cu] | | | 0.3-10 | mM | continuous |
| Ligand | | | TPMA, Me6TREN | | discrete |
| Type | | | | | |
| Buffer | PBS | | | | |
| Type | | | | | |
| Initiator | iBBr | | | | |
| Type | | | | | |
| [M] | | | 50, 100, 150, | mM | discrete |
| | | | 200, 250, 300, | | |
| | | | 350, 400 | | |
| Monomer | | | AAm, DMAAm, | | discrete |
| | | | HEAAm, AMP, | | |
| | | | NIPAAm, DEAAm, | | |
| | | | Tris AAm, | | |
| | | | qNAAm, | | |
| | | | DMAPAAm, | | |
| | | | AMPSA, SBAAm, | | |
| | | | CBAAm | | |
| [I] | 1 | mM | | | |
| Light mW | 15 | mW | | | |
| [NaCl] | | | 0-100 | mM | |
| Copper | CuCl | | | | |
| Type | | | | | |
| Rxn Time | | | 30-240 | min | continuous |

This high-throughput polymer conjugation platform was applied on several industrially relevant enzymes and processes. High-throughput bioconjugation screening was carried out with Lipase Type A from Candida Antarctica, using the twelve selected monomer types (FIG. 8) and three levels of initiator modification density-minimal (<5 Br/Lipase), partial (5-10 Br/Lipase), and maximum (>10 Br/Lipase). 400+ conjugates of a single enzyme were generated and their activity was compared to unmodified lipase (FIG. 9). Under ambient conditions (aqueous buffer, 30° C.), a significant number of conjugates showed retained activity (≥80%), FIG. 9. Lipase CAL A-polymer conjugates with high retained activity were predominately generated from partial and maximum levels of initiator density, zwitterionic polymers (pCBAAm, pSBAAm), anionic polymers (pAMPSA), hydrophilic polymers (pAAm, pHEAAm), or polymers with temperature-responsive properties (pNIPAAm, pDEAAm).

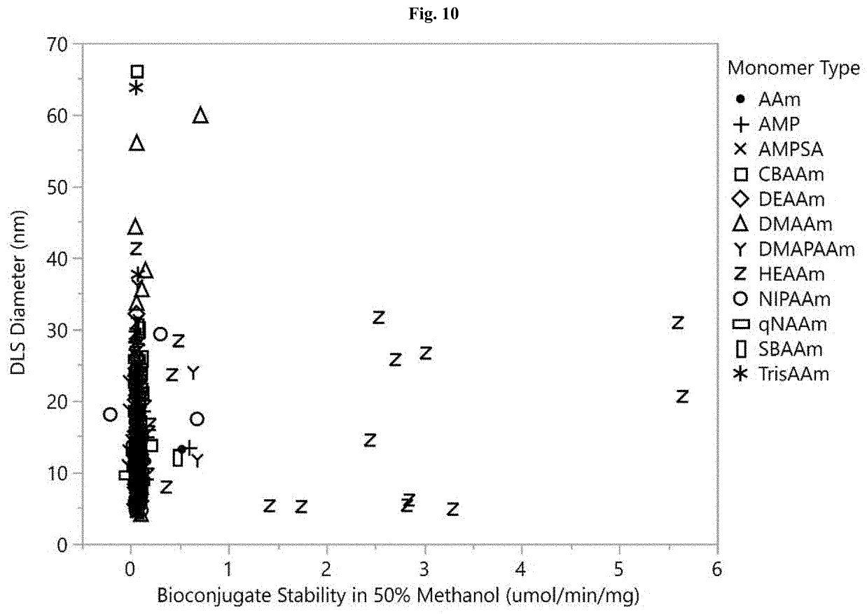

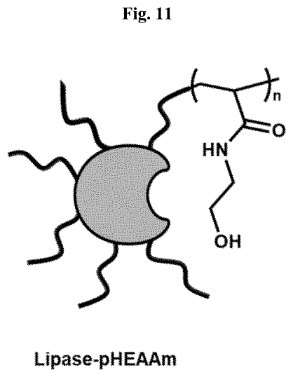

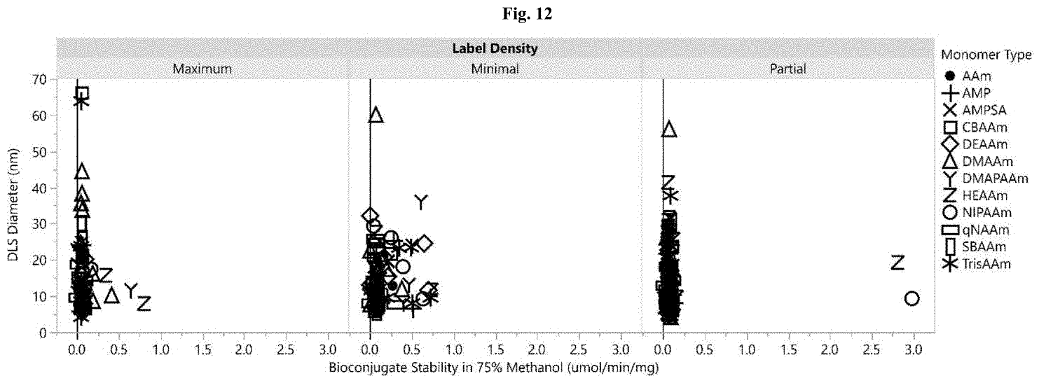

Samples (0.5 mg/ml) were incubated in 200 ul of 50% or 75% methanol solutions for 24 hours at 30° C. and shaken at 200 RPM. pNPB kinetic assays in aqueous environment with alcohol content less than 1% by volume were then conducted on the bio-conjugates following alcohol incubation, as well as unmodified Lipase Type A and negative controls of BSA and buffer. When unmodified lipase was exposed to 50-75% alcohol solutions, a complete loss of activity was observed. This screening revealed that for each alcohol type there were polymers that provided both stability and high activity to the enzyme. Utilizing the platform described herein, it was discovered that growing poly(N-hydroxyethyl acrylamide) (pHEAAm) from the enzyme provides complete protection against irreversible denaturation by methanol (FIG. 10, 11). The best performing conjugates are summarized in the Table 6. Selected lipase CALA-pHEAAm conjugates were also tested if they were active in the presence of methanol after incubation in 50% methanol solution. These samples were evaluated if they preserved their hydrolytic activity on pNPB hydrolysis in 50% by volume methanol solution in PBS (Table 6, entries 2-4). Other bio-conjugates retained at least 20% of Lipase Type A activity after incubation in 75% methanol (FIG. 12); the contributing polymers primarily being pDMAAm, pTRISAAm, pHEAAm, and pNIPAAm at minimal labeling density and pNIPAAm and pHEAAm at partial labeling density.

TABLE 6
Summary of lipase CALA-conjugates stable in 50% methanol solution in PBS.

| | | | | | | | | | 50% | 50% |
|---|---|---|---|---|---|---|---|---|---|---|
| | | | | | | | | Aqueous | MeOH | MeOH |
| | N of | | | Reaction | | | | Activity, | Stability, | Activity, |
| | initiating | Polymer | Ligand | time, | [NaCl], | Apparent | | μmol/ | μmol/ | μmol/ |
| | moieties | type | type | min | mM | Mn | PDI | (min · mg) | (min · mg) | (min · mg) |
| 1 | 8.2 | HEAAm | TPMA | 30 | 100 | 972,650 | 1.28 | 3.8 | 2.8 | |
| 2 | 8.2 | HEAAm | TPMA | 60 | 100 | 958,305 | 1.33 | 3.2 | 5.3 | 6.3 |
| 3 | 8.2 | HEAAm | TPMA | 90 | 100 | 1,009,784 | 1.33 | 3.6 | 5.0 | 4.9 |
| 4 | 14.6 | DMAAm | TPMA | 30 | 0 | 707,644 | 1.32 | 2.0 | 1.42 | 1.62 |
| 5 | 8.2 | HEAAm | TPMA | 90 | 100 | 919,633 | 1.35 | 3.7 | 3.3 | |
| 6 | 8.2 | HEAAm | Me₆TREN | 30 | 50 | 893,967 | 1.36 | 3.6 | 3.0 | |
| 7 | 8.2 | HEAAm | Me₆TREN | 60 | 50 | 883,273 | 1.34 | 4.5 | 2.4 | |
| 8 | 8.2 | HEAAm | Me₆TREN | 120 | 50 | 898,402 | 1.38 | 3.7 | 2.7 | |
| 9 | 8.2 | HEAAm | Me₆TREN | 240 | 50 | 926,376 | 1.39 | 3.6 | 2.8 | |
| 10 | 7.9 | HEAAm | Me₆TREN | 30 | 50 | 191,893 | 1.74 | 1.7 | 5.6 | 1.6 |

Reaction conditions: [Cu]=0.3 mM, [TEA]=100 mM, buffer type—PBS, except for entry 4—HEPES, [M]=400 mM, except for entry 4 and 10-50 mM, blue LED light at 15 mW was used for photo-irradiation; number average molecular weight Mn and polydispersity (PDI) was determined by running sample on aqueous GPC (Agilent AdvanceBio 300 column 7×150 mm, PBS, pH˜7.4, 30° C., protein standards calibration); aqueous activity was measured upon formation and purification of bioconjugate in pNPB hydrolysis assay in PBS at 30° C.; 50% methanol stability was measured as activity in aqueous pNPB hydrolysis after incubation of the lipase-polymer conjugate in 50% by volume methanol solution in PBS for 24 hours at 30° C.; 50% methanol activity was measured as activity in pNPB hydrolysis in 50% by volume methanol in PBS after incubation of the lipase-polymer conjugate in 50% by volume methanol solution in PBS for 24 hours at 30° C.

### Example 4

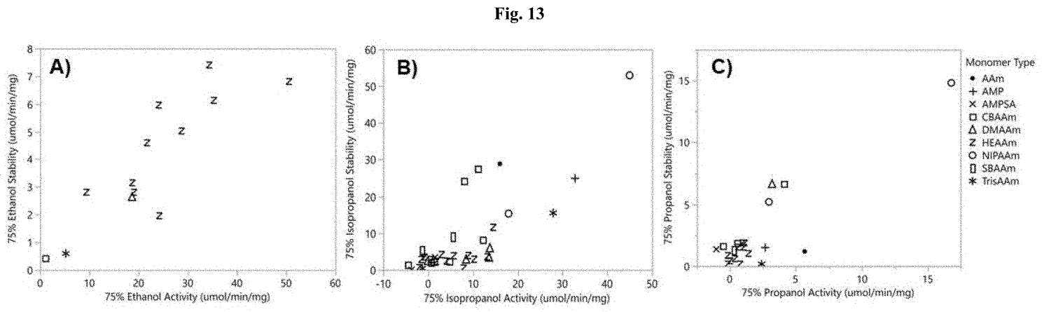

The set of Lipase Type A bio-conjugates that passed the initial screening of incubation in 50% and 75% methanol were subjected to further testing by incubation in 50% methanol, ethanol, 2-propanol, and 1-propanol at 30° C., 200 RPM for 24 hours. Those bioconjugates that retained at least 1 mol/min/mg of hydrolysis activity in the subsequent pNPB assays after incubation in alcohols were further tested by incubation in 75% solution of the same alcohol. The bio-conjugates were then tested in the standard pNPB kinetic assay and in an adapted end-point assay on the pNPB substrate but wherein the assay solution itself was 75% of the corresponding alcohol (FIG. 13). As with methanol incubation, pHEAAm demonstrated the best retained activity when incubated in ethanol (FIG. 13, panel A). For incubation in 75% 2-propanol (FIG. 13, panel B) and 1-propanol (FIG. 13, panel C), a selection of polymers that conveyed good hydrolysis activity retention was observed, including pNIPAAm, pCBAAm, pAMP, pSBAAm, and pTRISAm. Results and polymerization conditions for the best performing conjugates are summarized in the Table 7. For isopropanol and n-propanol the activity increase was as high as 10- and 30-fold respectively for lipase-poly(N-isopropylacrylamide) (pNIPAAm).

TABLE 7
Summary of lipase CALA-conjugates stable in 75% alcohol solutions in PBS.

| | N of | | Reaction | | | | | Stability in 75% alcohol | | |
|---|---|---|---|---|---|---|---|---|---|---|
| | initiating | Polymer | time, | [NaCl], | | | Hydrodynamic | solutions, μmol/(min · mg) | | |
| | moieties | type | min | mM | Mn | PDI | diameter, nm | EtOH | nPrOH | iPrOH |
| 1 | 14.6 | DMAAm | 30 | 0 | 707,645 | 1.32 | 16.0 | 2.6 | 6.6 | 6.0 |
| 2 | 8.2 | HEAAm | 90 | 100 | 919,633 | 1.35 | 4.9 | 6.8 | 1.5 | 2.8 |
| 3 | 8.2 | HEAAm | 30 | 50 | 893,967 | 1.36 | 26.7 | 5.0 | 1.5 | 4.2 |
| 4 | 8.2 | HEAAm | 60 | 50 | 883,273 | 1.34 | 14.5 | 6.1 | | 11.6 |
| 5 | 17.3 | TrisAAm | 30 | 0 | 41,824 | 1.19 | 63.8 | | | 15.5 |
| 6 | 17.3 | CBAAm | 30 | 0 | | | 15.1 | | 6.6 | 27.5 |
| 7 | 7.9 | AMP | 30 | 0 | 29,702 | 1.20 | 10.4 | | 1.5 | 25.0 |
| 8 | 7.9 | NIPAAm | 30 | 0 | | | 9.3 | | 14.8 | 52.9 |
| 9 | 7.9 | SBAAm | 30 | 0 | 45,189 | 1.24 | 30.4 | | | 9.0 |
| 10 | 4.1 | SBAAm | 30 | 50 | 633,121 | 1.24 | 12.1 | | 1.3 | 5.3 |
| 11 | 4.1 | CBAAm | 30 | 100 | | | 20.4 | | | 8.2 |

Reaction conditions: [Cu]=0.3 mM, [TEA]=100 mM; reaction medium was HEPES buffer, except entries 2-4, 10-11 used PBS as reaction medium; monomer concentration was 50 mM, except entries 2-4, 10-11 used 400 mM; ligand type was TPMA, except entries 3,4, 10 used Me₆TREN. Number average molecular weight Mn and polydispersity (PDI) was determined by running sample on aqueous GPC (Agilent AdvanceBio 300 column 7×150 mm, PBS, pH˜7.4, 30° C., protein standards calibration); hydrodynamic diameter was determined by dynamic light scattering method; 75% alcohol stability was measured as activity in aqueous pNPB hydrolysis after incubation of the lipase-polymer conjugate in 75% by volume alcohol solution in PBS for 24 hours at 30° C.

### Example 5

The best performing candidates were tested in industrially relevant biotransformation reactions to validate the predictive power of the synthesis and screening process described herein. Lipase CALA-pHEAAm was tested in methyl oleate synthesis reaction to demonstrate methanol stabilization properties in industrially relevant esterification reactions:

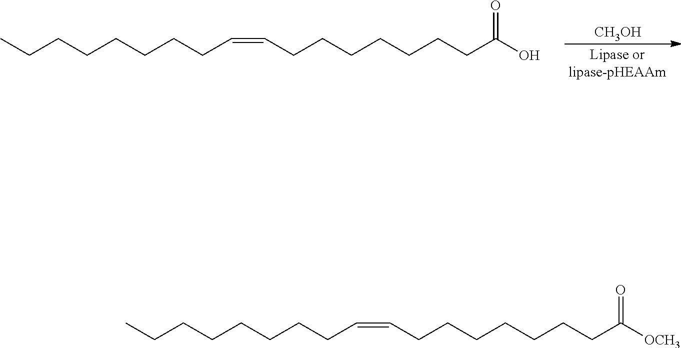 [SMILES: CCCCCCCC/C=C\CCCCCCCC(=O)O ; "CH3OH" ; CCCCCCCC/C=C\CCCCCCCC(=O)OC]

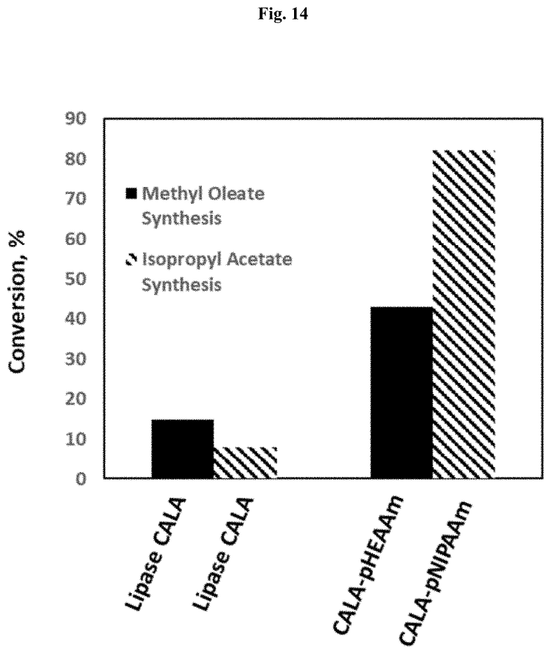

The esterification activity of the lipase and its polymer conjugate was investigated with a reaction converting oleic acid to fatty acid esters, where the full amount of alcohol reagent was provided at the beginning of the reaction. To 20 g of oleic acid was added 10 mg of lipase or lipase-polymer conjugate, then 5.7 mL of methanol (2 equivalents to oleic acid) was added before incubating 24 hours at 40° C. The conversion of oleic acid to methyl oleate was calculated by an endpoint titration of remaining oleic acid. Such addition of alcohol is typically very damaging to the enzyme, which was confirmed by low conversion of this reaction for unmodified enzyme (15%). When lipase-polymer conjugate was applied however, the yield of the reaction increased 3-fold as a result of the polymer imparted methanol stability (FIG. 14).

### Example 6

Lipase CALA-pNIPAAm was tested in isopropyl acetate synthesis reactions (FIG. 14) to demonstrate isopropanol stabilization properties in industrially relevant transesterification reactions. Transesterification activity was tested by the reaction of vinyl acetate with isopropanol to produce isopropyl acetate:

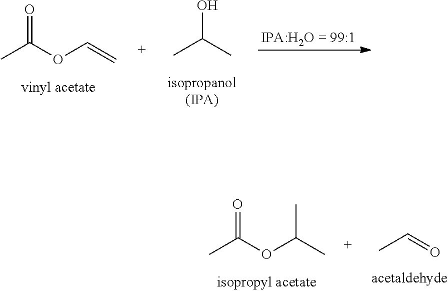

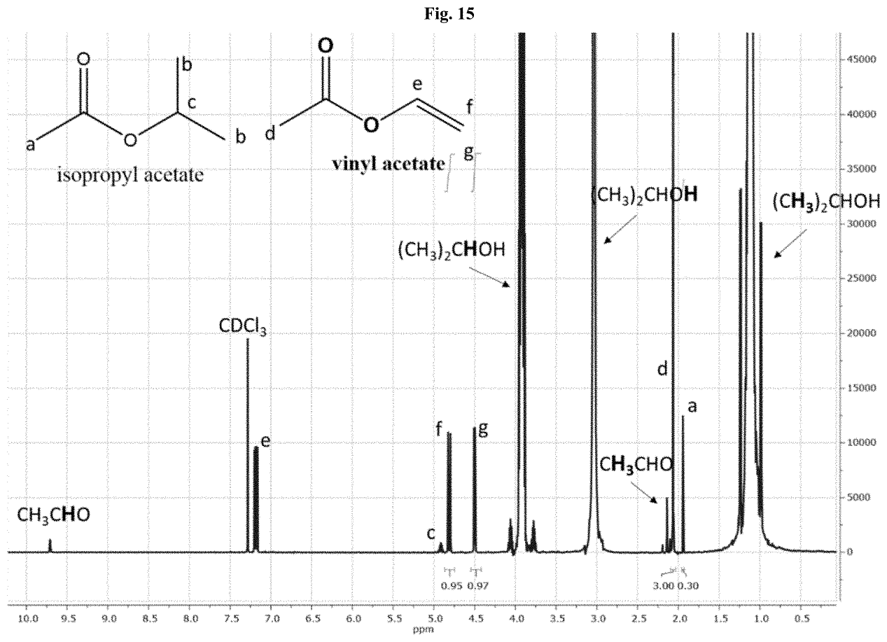

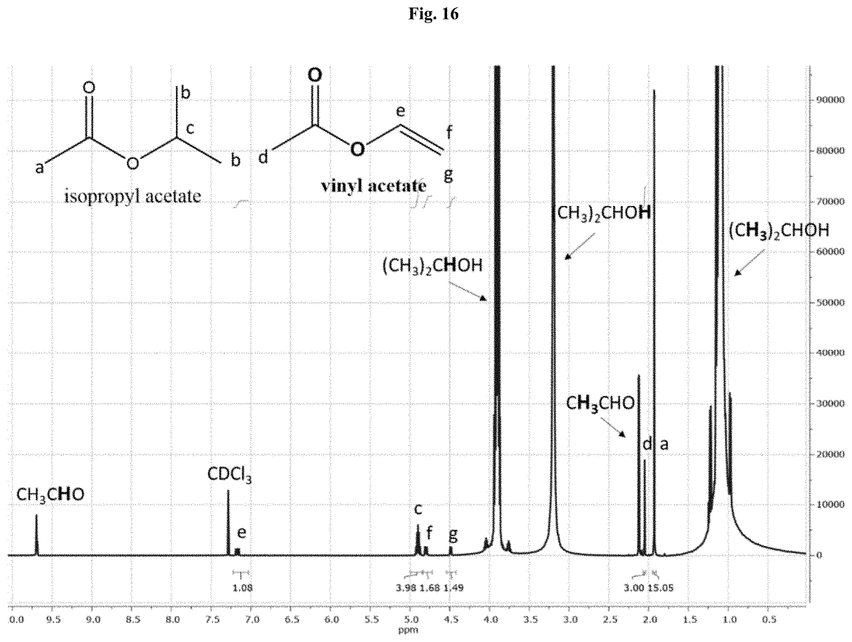

Lyophilized lipase CALA and CALA-pNIPAAm were resuspended in 300 mM vinyl acetate in isopropanol (1% water v/v) and stirred at room temperature for 90 hours. Conversion of vinyl acetate to isopropyl acetate was measured by NMR (FIGS. 15 and 16). The reaction catalyzed by unmodified lipase only reached 8% conversion, while the reaction catalyzed by its conjugate with pNIPAAm reached 82% conversion. Such findings have never been reported before and could have a significant impact for biocatalysis involving alcohols.

### Example 7

In another example, lipases of different types can be used to catalyze (trans)esterification of fatty acids and tri-, di-, and mono-glycerides of fatty acids to provide fatty acid methyl ester (FAME) or biodiesel. Lipase types include but are not limited to lipase type B from Candida Antarctica and lipase from Thermomyces lanuginosus. Biodiesel is commercially produced through either the chemical or enzymatic conversion of soy bean oil (among other oils) to FAME. Biodiesel feedstocks can include refined soybean or canola oil, distillers corn oil, yellow or brown grease, and others. Current enzymatic processes for biodiesel production are limited by enzymatic intolerance to methanol, a necessary reagent for FAME production. In order to overcome this enzymatic instability, current biodiesel production requires slow addition of methanol, resulting in long reaction times to keep methanol concentrations low. This long reaction time is economically prohibitive, reducing biodiesel output rates. It can be expected that lipases modified with polymers such as pHEAAm or pHMAAm are more stable in the presence of methanol. Higher methanol amount is added to the reaction mixture at a faster rate, which leads to faster and more efficient conversion of oil-based feedstocks to biodiesel.

### Example 8

In another example, alcohol-stabilizing polymers can be grafted from other classes of enzymes to confer similar alcohol stabilization properties. Transaminases, reductive aminases, ketoreductases, nitrilases, and glucose dehydrogenase can be modified with pHEAAm to be stable in methanol or ethanol. Or they can be modified with pNIPAAm or pAMP to be stable in n-propanol or isopropanol. In one such example, ketoreductase is modified with pNIPAAm to impart isopropanol stability for production of acetone by the oxidation of isopropanol and simultaneous regeneration of its co-factor, NADH. In this model reaction, isopropanol serves not only as the solvent, but also a reagent for the co-factor recycling reaction. Ketoreductase is modified with ATRP-initiator molecules and then purified by high-throughput vacuum assisted ultrafiltration using a multi-well plate vacuum manifold utilizing multi-well filter plates with 10 KDa or 30 KDa molecular weight cutoff membranes. The resulting enzyme-initiator conjugates are then analyzed for degree of modification and retained activity. Activity is measured by spectroscopically monitoring the reduction of NAD+. Conjugates retaining ≥80% activity are then selected for polymerization with NIPAAm monomers. The resulting ketoreductase-pNIPAAm conjugates are purified by centrifugal ultrafiltration with 10 KDa, 30 KDa or higher molecular weight cutoff ultrafiltration membranes, removing all small molecules including leftover monomer and catalyst. Conjugates are analyzed to determine size and molecular weight of the resulting enzyme-polymer conjugates, and to determine residual activity. The conjugates are expected to be stable in short chain alcohols such as isopropanol.

### Example 9

In another example, transaminase and transaminase-polymer conjugates asymmetrically aminate organic ketones such as acetophenone. Asymmetric transamination reactions are commonly used in pharmaceutical API synthesis and are often performed in short chain alcohol solvents. As such, stabilization of transaminase enzymes to alcohols is important for increasing API synthesis yields and efficiency. Transaminase is modified with ATRP-initiator molecules and then purified by high-throughput vacuum assisted ultrafiltration using a multi-well plate vacuum manifold utilizing multi-well filter plates with 10 KDa or 30 KDa molecular weight cutoff membranes. The resulting enzyme-initiator conjugates are then analyzed for degree of modification and retained activity. Activity is measured by spectroscopically monitoring the production of a colorimetric readout following amine donation from 4-Nitrophenethylamine hydrochloride. Conjugates retaining ≥80% activity are then selected for polymerization. The resulting enzyme-polymer conjugates are purified by centrifugal ultrafiltration with 10 KDa and 30 KDa molecular weight cutoff ultrafiltration membranes, removing all small molecules including leftover monomer and catalyst. Conjugates are analyzed to determine size and molecular weight of the resulting enzyme-polymer conjugates, and to determine residual activity. The conjugates are expected to be stable in short chain alcohols such as methanol and ethanol.

### Example 10

Other polymer with multiple hydroxyl groups can be grafted from an enzyme to provide stabilization in alcohols. For example, pHEMA, pHEA, or pHMAAm are grown from an enzyme by ATRP to provide stability in alcohol solutions. An enzyme is modified with ATRP-initiator molecules and then purified by high-throughput vacuum assisted ultrafiltration using a multi-well plate vacuum manifold utilizing multi-well filter plates with 10 KDa or 30 KDa molecular weight cutoff membranes. The resulting enzyme-initiator conjugates are then analyzed for degree of modification and retained activity. Activity is measured by spectroscopically. Conjugates retaining ≥80% activity are then selected for polymerization with HEMA, HEA, or pHMAAm monomers. The resulting enzyme-pHEMA, -pHEA, or -pHMAAm conjugates are purified by centrifugal ultrafiltration with 10 KDa and 30 KDa molecular weight cutoff ultrafiltration membranes, removing all small molecules including leftover monomer and catalyst. Conjugates are analyzed to determine size and molecular weight of the resulting enzyme-polymer conjugates, and to determine residual activity. The conjugates are expected to be stable in short chain alcohols such as methanol and ethanol.

1. An enzyme-polymer conjugate comprising:
at least one polymer covalently bound to an enzyme via a covalent linker;
wherein the at least one polymer is selected from pHEAAm, pDMAAm, pTRISAAm, pNIPAAm, pCBAAm, pSBAAm, pAMPSA, pAMP, pAAm and pDEAAm, or a combination thereof; and
wherein the enzyme is a lipase.

2.-3. (canceled)

4. The conjugate of claim 1, wherein the at least one polymer is covalently bound to a lysine residue of the enzyme via the covalent linker.

5. The conjugate of claim 1, wherein the covalent linker comprises:

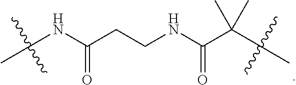 [SMILES: *NC(=O)CCNC(=O)C(*)(C)C]

6. The conjugate of claim 1, wherein at least 30% of the total lysine residues of the enzyme are conjugated to a polymer.

7. The conjugate of claim 1, wherein the conjugate comprises a plurality of polymers, wherein the plurality of polymers is made by growing the polymers directly from the surface of the enzyme using atom-transfer radical polymerization (ATRP).

8. (canceled)

9. The conjugate of claim 7, wherein each polymer in the plurality of polymers comprises monomeric units of the same type.

10. The conjugate of claim 7, wherein each polymer in the plurality of polymers comprises a first polymer and a second polymer, wherein the first polymer and the second polymer each comprise monomeric units of a different type.

11. The conjugate of claim 1, wherein the at least one polymer comprises 10 to 200 monomeric units.

12. (canceled)

13. The conjugate of claim 1, wherein the conjugate exhibits at least 1 μmol/min/mg of enzymatic activity after 1 hour of incubation in an aqueous solution comprising at least 50% by volume of a C₁₋₃ alcohol.

14. (canceled)

15. A method of catalyzing a reaction, comprising:
combining an enzyme-polymer conjugate with a substrate for an enzyme in an aqueous solution comprising at least 20% by volume of a C₁₋₆ alcohol, thereby forming a reaction mixture;
wherein the enzyme-polymer conjugate comprises at least one polymer covalently bound to the enzyme via a covalent linker, wherein the at least one polymer is selected from pHEAAm, pDMAAm, pTRISAAm, pNIPAAm, pCBAAm, pSBAAm, pAMPSA, pAMP, pAAm and pDEAAm, or a combination thereof, and wherein the enzyme is an esterase, lipase, transferase, oxidoreductase or protease.

16. The method of claim 15, further comprising separating a product from the reaction mixture.

17-23. (canceled)

24. The method of claim 15, wherein the at least one polymer is covalently bound to a lysine residue of the enzyme via a covalent linker.

25. The method of claim 15, wherein the covalent linker comprises:

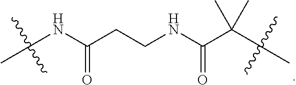 [SMILES: *NC(=O)CCNC(=O)C(*)(C)C]

26. The method of claim 15, wherein at least 30% of the total lysine residues of the enzyme are conjugated to a polymer.

27. The method of claim 15, wherein the conjugate comprises a plurality of polymers, wherein the plurality of polymers is made by growing the polymers directly from the surface of the enzyme using atom-transfer radical polymerization (ATRP).

28. (canceled)

29. The method of claim 27, wherein each polymer in the plurality of polymers comprises monomeric units of the same type.

30. The method of claim 27, wherein each polymer in the plurality of polymers comprises a first polymer and a second polymer, wherein the first polymer and the second polymer each comprise monomeric units of a different type.

31. The method of claim 15, wherein the at least one polymer comprises 10 to 200 monomeric units.

32-34. (canceled)

35. The method of claim 15, wherein the conjugate exhibits at least 1 μmol/min/mg of enzymatic activity.

36. A solution comprising:
an aliphatic alcohol, wherein the alcohol comprises no greater than 75 percent water by volume as a co-solvent; and
an enzyme-polymer conjugate that retains at least 30 percent of the activity of the native enzyme in water;
wherein the enzyme-polymer conjugate comprises covalently bound polymer chains grown from an enzyme-initiator conjugate.

37-50. (canceled)
